(19) United States
(12) Patent Application Publication
(10) Pub. No.: US 2007/0105085 A1
(43) Pub. Date: May 10, 2007

(54) Crystal of a transporter-ligand complex and methods of use

(75) Inventors: Geoffrey Chang, La Jolla, CA (US); Christopher L. Reyes, San Diego, CA (US); Andrew Ward, San Diego, CA (US); Jodie Yu, San Diego, CA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: The Scripps Research Institute

(21) Appl. No.: 11/432,967

(22) Filed: May 12, 2006

Related U.S. Application Data
(60) Provisional application No. 60/680,401, filed on May 12, 2005.

(51) Int. Cl. C12Q 1/00; G06F 19/00

(52) U.S. Cl. 435/4; 702/19

(57) ABSTRACT

The invention relates to the three-dimensional structure of a crystal of a transporter protein complexed with a ligand. The three-dimensional structure of the transporter-ligand complex is disclosed. The transporter-ligand crystal structure, wherein the ligand is an inhibitor molecule, is useful for providing structural information that may be integrated into drug screening and drug design processes. Thus, the invention also relates to methods for utilizing the crystal structure of the transporter-ligand complex for identifying, designing, selecting, or testing inhibitors of the transporter protein, such inhibitors being useful as therapeutics for the treatment or modulation of i) diseases; ii) disease symptoms; or iii) the effect of other physiological events mediated by the transporter.

### CROSS-REFERENCE TO RELATED APPLICATIONS

This application claims priority from U.S. Provisional Application Ser. No. 60/680,401 filed on May 12, 2005, which is incorporated herein by reference in its entirety.

### STATEMENT REGARDING FEDERALLY SPONSORED RESEARCH OR DEVELOPMENT

This invention was made in part with Government support under National Institutes of Health Grant GM61905 The Government has certain rights in the invention.

### INCORPORATION-BY-REFERENCE OF MATERIAL SUBMITTED ON A COMPACT DISC

The Compact Disc Appendix (CD Appendix), which is a part of the present disclosure, includes one folder designated CD Appendix on the compact disc. The CD Appendix contains “Table 1 (256 pages).DOC” of 2.62 MB and “Table 2 (226 pages).DOC” of 2.28 MB, tables having over 50 pages, of the present invention comprising the atomic coordinates of exemplary crystal structures. A portion of the disclosure of this patent document contains material which is subject to copyright protection. The copyright owner of that material has no objection to the facsimile reproduction by anyone of the patent document or the patent disclosure, as it appears in the Patent and Trademark Office patent files or records, but otherwise reserves all copyright. The subject matter of the CD Appendix is incorporated herein by reference in its entirety.

The Sequence Listing, which is a part of the present disclosure, includes a computer readable form and a written sequence listing comprising nucleotide and amino acid sequences of the present invention. The sequence listing information recorded in computer readable form is identical to the written sequence listing. The subject matter of the Sequence Listing is incorporated herein by reference in its entirety.

### FIELD

The present invention relates to a three dimensional structure of an Adenosine Triphosphate Binding Cassette Transporter (“ABC Transporter”) complexed with its ligand (“Transporter-Ligand Complex”), and in particular ABC Transporter MsbA (“MsbA”) complexed with lipopolysaccharide (“LPS”) and nucleotide, MsbA complexed with PSC-833 or related compound and nucleotide, three dimensional coordinates of a Transporter-Ligand Complex, models thereof, and uses of such structures and models.

### INTRODUCTION

A major cause of antibiotic and cancer drug resistance is attributed to a robust array of multi-drug resistant (“MDR”) transporters that extrude drug compounds out of the cell. MDR transporters can be divided into two classes based on their source of energy: Secondary transporters, which use proton gradients to facilitate an antiporter mechanism, and ABC transporters that couple the hydrolysis of ATP to substrate transport across the cell membrane. ABC transporters belong to one of the largest superfamilies of proteins and that either import or export a broad range of substrates that include amino acids, ions, sugars, lipids, and drugs. The differences in their substrate specificities are reflected in their overall divergence in their transmembrane domains (TMDs). While bacteria genomes encode both classes of ABC transporters, eukaryotes have only exporters suggesting an early evolutionary divergence of their TMDs. In humans, 46 ABC transporters have been identified and play important roles in human diseases, which include cystic fibrosis, macular dystrophy, and several neurological disorders.

All ABC transporters are composed minimally of two nucleotide binding domains (“NBDs”) and two TMDs. The NBD, which is also called an ABC, have been multiply determined and are similar across eukaryotes and prokaryotes. The TMD recognizes and mediates the passage of substrates across the cell membrane including the removal of a large number of chemically unrelated lipids and toxins directly from the cell membrane. Many of the ABC Transporters are believed to translocate useful cytotoxins such as anti-cancer drugs in addition to lipids and toxins leading to multi-drug resistance.

A widely studied human MDR-ABC transporter is the P-glycoprotein or human MDR1/ABCB1. However, the specific mechanism and structural basis for ATP hydrolysis for most ABC transporters is unknown. Studies in vitro demonstrate that MsbA is an ATPase that is specifically stimulated by lipid A. Loss of MsbA from the cell membrane or mutations that disrupt transport results in the lethal accumulation of lipid A in the inner cell membrane. MsbA is the only essential ABC Transporter in prokaryotes and is conserved in every bacterium with more than 30 orthologs identified. MsbA is a bacterial homolog of human MDR1/ABCB1 by protein sequence homology and has overlapping substrate specificities with the MDR-ABC Transporter LmrA from Lactococcus lactis.

Despite attempts to model the structural changes of MsbA and other MDR-ABC transporters, a detailed view of conformational rearrangements during ATP hydrolysis and substrate translocation has remained elusive. Thus, the development of useful reagents for treatment or diagnosis of disease was hindered by lack of structural information of ABC Transporters, and in particular Transporter-Ligand Complexes. Therefore, there is a need in the art to elucidate the three dimensional structure and models of Transporter-Ligand Complexes, and to use such structures and models in therapeutic strategies, such as drug design.

### SUMMARY

This invention provides a method for for designing a drug which interferes with an activity of an Adenosine Triphosphate Binding Cassette Transporter, the method comprising (a) providing on a digital computer a three-dimensional structure of a Transporter Ligand Complex comprising the Adenosine Triphosphate Binding Cassette Transporter and at least one ligand of the Transporter; and (b) using software comprised by the digital computer to design a chemical compound which is predicted to bind to the Adenosine Triphosphate Binding Cassette Transporter. The method may further comprise (c) synthesizing the chemical compound; and (d) evaluating the chemical compound for an ability to interfere with an activity of the Adenosine Triphosphate Binding Cassette Transporter.

In various aspects of the invention, the chemical compound is designed by computational interaction with reference to a three dimensional site of the structure of the Transporter-Ligand Complex, wherein the three dimensional site is selected from the group consisting of TM1: 37-44, TM2: 57-78, TM6: 277-291, EC1: 49-68, EC2: 160-168, EC3: 270-278, ICD1: 92-142, elbow helix: 10-22, TM1: 26-37, TM3: 143-153, TM4: 180-187, TM5: 251-256, TM6: 291-303, and any combination thereof.

In various aspects of the invention, the chemican compound is designed by computational interaction with reference to a three dimensional site of the structure of the Transporter-Ligand Complex, wherein the three dimensional site is selected from the group consisting of LSGGQ, A-loop, RXYD, ICD1 and a combination thereof. In yet another aspect, the LSGGQ site can comprise L481. In another aspect, the A-loop site can comprise at least one amino acid selected from the group consisting of F349, Y351, P352, G353, R354 and E355. In another aspect, the RXYD site can comprise at least one amino acid selected from the group consisting of R391, F392, Y393, D394, 1395 and D396. In yet another aspect, the ICD1 site can comprise at least one amino acid selected from the group consisting of M108 and F115.

In accordance with a further aspect of the invention, a method is provided for generating a model of a three dimensional structure of a Transporter-Ligand Complex, the method comprising (a) providing an amino acid sequence of a known Transporter-Ligand Complex and an amino acid sequence of a target Transporter-Ligand Complex; (b) identifying structurally conserved regions shared between the known Transporter-Ligand Complex amino acid sequence and the target Transporter-Ligand Complex amino acid sequence; and (c) assigning atomic coordinates from the conserved regions to the target Transporter-Ligand Complex. In various aspects, the known Transporter-Ligand Complex has a three dimensional structure described by atomic coordinates that substantially conform to atomic coordinates set forth in Table 1 or Table 2.

In accordance with a further aspect of the invention, a method is provided for determining a three dimensional structure of a target Transporter-Ligand Complex structure comprising (a) providing an amino acid sequence of a target structure, wherein the three dimensional structure of the target structure is not known; (b) predicting the pattern of folding of the amino acid sequence in a three dimensional conformation using a fold recognition algorithm; and (c) comparing the pattern of folding of the target structure amino acid sequence with the three dimensional structure of a known Transporter-Ligand Complex. In various aspects, the known Transporter-Ligand Complex comprises a three dimensional structure described by atomic coordinates that substantially conform to atomic coordinates set forth in Table 1 or Table 2.

In various embodiments, the present teachings disclose compositions and methods for treating a microbial infection. A composition of these embodiments can comprise an inhibitor of an ATP-binding cassette transporter, and an antibiotic. In some alternative embodiments, a composition for treating a microbial infection can comprise an anti-microbial agent covalently attached to an antibody directed against an ATP-binding cassette transporter. In yet other alternative embodiments, a composition for treating a microbial infection can comprise an inhibitor of an ATP-binding cassette transporter covalently attached to an anti-microbial agent. Methods of these teachings include administration of a composition as described herein to a patient in need thereof.

Accordingly, in various configurations of these embodiments, the present teachings further disclose that an inhibitor for treating a microbial infection can be selected by performing a rational drug design with a three-dimensional structure determined for a crystal of the ATP-binding cassette. In certain aspects, an inhibitor can be selected by a) performing a rational drug design with a three-dimensional structure determined for a crystal of the ATP-binding cassette to identify a candidate inhibitor; b) contacting the candidate inhibitor with the ATP-binding cassette transporter; and c) detecting inhibition of at least one activity of the transporter. In various aspects, the ATP-binding transporter can be a prokaryotic ATP-binding transporter or a eukaryotic transporter. A prokaryotic ATP-binding transporter can be a bacterial ATP-binding transporter, such as a gram negative bacterial ATP-binding transporter or a gram positive bacterial ATP-binding transporter. A gram negative bacterial ATP-binding transporter can be from any gram negative bacteria, such as Salmonella or E. coli. Accordingly, a Salmonella ATP-binding transporter can be an ATP-binding transporter such as a Salmonella typhimurium msbA ATP-binding transporter.

In various configurations of these embodiments, an inhibitor can be an antibody directed against an ATP-binding cassette transporter such as a against the Salmonella typhimurium msbA ATP-binding cassette transporter. In various aspects, an antibody can be a polyclonal or a monoclonal antibody. An antibody can also be directed against particular structures, portions, or domains of an ATP-binding cassette transporter, such as an extracellular loop domain a substrate contacting domain, intracellular domain, a drug-binding domain, or an extracellular domain. In various aspects of these configurations, an extracellular loop domain can comprise a peptide sequence such as kpllddgfgktdrsvllwmp (SEQ ID NO: 1), mfyyswqls (SEQ ID NO: 2) or asfpsvmds (SEQ ID NO: 3); a domain which contacts a substrate can comprise a peptide sequence such as naasdtfm (SEQ ID NO: 4), gktdrsvllwmplvviglmilr (SEQ ID NO: 5) or dsltagtitvvfssm (SEQ ID NO: 6); an intracellular domain of the ABC transporter can comprise a sequence vsgkvvmtmrrrlfghmmgmpvaffdkqstgtllsritydseqvassssga (SEQ ID NO: 7); a drug-binding domain can comprise a peptide sequence such as wqtfrrlwptiap (SEQ ID NO: 8); glivagialiln (SEQ ID NO: 9); litvvregasi (SEQ ID NO: 10); iairvvsk (SEQ ID NO: 11); sdpiiq (SEQ ID NO: 12) or mialmrplksltn (SEQ ID NO: 13); and an extracellular domain of the ABC transporter can comprise a peptide sequence such as ddgfgktdrsvl (SEQ ID NO: 14), wql (SEQ ID NO: 15), or psvmdslt (SEQ ID NO: 16).

Certain configurations of these embodiments involve compositions and methods for detecting inhibition of one or more activities of an ATP-binding transporter. In certain aspects, detecting inhibition of an activity can include detecting inhibition of an activity such as ATP hydrolysis, transmembrane transport of a cationic hydrophobic compound, binding of the cationic hydrophobic compound to the transporter or release of the cationic hydrophobic compound from the transporter. In these configurations, a cationic hydrophobic compound can be a lipopolysaccharide, such as an Ra rough type lipopolysaccharide, an Rb rough type lipopolysaccharide, an Rc rough type lipopolysaccharide, an Rd rough type lipopolysaccharide and an Re rough type lipopolysaccharide.

In embodiments of the present teachings which comprise an anti-microbial agent covalently attached to an antibody directed against an ATP-binding cassette transporter, an anti-microbial agent can be any anti-microbial agent known to persons of skill in the art which can be attached or coupled to an antibody, such as, in non-limiting example, an anti-microbial peptide. In non-limiting example, an anti-microbial peptide can be an insect anti-microbial peptide, such as a cecropin, including a hybrid cecropin such as a cecropin-melittin hybrid peptide. In alternative configurations, an anti-microbial peptide can be from a vertebrate animal including a mammal. Some non-limiting examples of anti-microbial peptides which can be used in these embodiments include a myeloid antimicrobial peptide, an alpha-defensin, a beta-defensin, a protegrin, a porcine cecropin P1, a Bac5, a Bac7, a PR-39 and a prophenin

Certain embodiments of the present teachings include compositions and methods for treating a cancer. A composition of these teachings can comprise an inhibitor of an ATP-binding cassette transporter and an anticancer chemotherapeutic. Methods of these teachings include administration of a composition as described herein to a patient in need thereof.

Accordingly, in various configurations, the inhibitor can be selected as described above. Similarly, an inhibitor can inhibit at least one activity of a transporter. The activity inhibited can be, in non-limiting example, ATP hydrolysis, transmembrane transport of a cationic hydrophobic compound, binding of the cationic hydrophobic compound to the transporter or release of a cationic hydrophobic compound from the transporter. In various aspects, the ATP-binding transporter can be a eukaryotic ATP-binding transporter, such as a mammalian ATP-binding transporter, in particular a human ATP-binding transporter. A human ATP-binding transporter can be, in non-limiting example, a P-glycoprotein ATP-binding transporter such as an MDR1 ATP-binding transporter. Furthermore, an antibody as used herein can be a polyclonal antibody or a monoclonal antibody.

In various aspects, an antibody can be directed against an entire ATP-binding transporter, or at least one region, domain, or subsequence thereof, such as, for example, an extracellular domain such as an extracellular loop domain, a domain which contacts a substrate, an intracellular domain, or a drug-binding domain. Furthermore, an anticancer chemotherapeutic can be a cationic hydrophobic compound. Non-limiting examples of anticancer chemotherapeutics include colchicine, doxorubicin, adriamycin, vinblastine, digoxin, saquinivir and paclitaxel. In some configurations, the inhibitor of an ATP-binding cassette transporter and the anticancer chemotherapeutic can be covalently attached. An antibody, as used herein, can comprise an intact antibody molecule, or a fragment or variant thereof, such as, for example, an Fab fragment or a phage display antibody.

The invention also provides a Transporter-Ligand Complex in a crystalline form. In various aspects, the Transporter-Ligand Complex comprises MsbA, LPS and a nucleotide. The complex may be arranged in a space group C2 so as to form a unit cell of dimensions a=271.4 Å, b=122.0 Å, c=121.7 Å, α=90°, β=121.7°, γ=90°. According to various aspects, the MsbA is Salmonella typhimurium MsbA. In various aspects, the crystal is sufficiently pure to determine atomic coordinates of the ligand-complexed MsbA by X-ray diffraction to a resolution of about 4.2 Å.

The invention also provides a complex wherein the Transporter-Ligand Complex comprises MsbA and a cyclosporin D analogue. The cyclosporin D analogue can be PSC-833 and may further comprise a nucleotide, for example, ADP. In another aspect, the complex can be arranged in a space group C2 so as to form a unit cell of dimensions a=268.2 Å, b=121.4 Å, c=176.2 Å, β=121.2°. The crystal can be sufficiently pure to determine atomic coordinates of the ligand-complexed MsbA by X-ray diffraction to a resolution of about 4.5 Å, and in another aspect, 0.4.2 Å.

In accordance with a further aspect of the invention, a therapeutic composition is provided comprising one or more therapeutic compounds, the therapeutic compound being capable of reducing MDR that involves an Adenosine Triphosphate Binding Cassette Transporter. The therapeutic compound can be designed using structure based drug design.

In accordance with yet another aspect of the invention, a three dimensional computer image of the three dimensional structure of a Transporter-Ligand Complex, wherein the structure substantially conforms with the three dimensional coordinates listed in Table 1 or Table 2. Also provided is a computer-readable medium encoded with a set of three dimensional coordinates represented in Table 1 or Table 2, wherein, using a graphical display software program, the three dimensional coordinates create an electronic file that can be visualized on a computer capable of representing said electronic file as a three dimensional image. In addition, a computer-readable medium is provided which is encoded with a set of three dimensional coordinates of a three dimensional structure which substantially conforms to the three dimensional coordinates represented in Table 1 or Table 2, wherein, using a graphical display software program, the set of three dimensional coordinates create an electronic file that can be visualized on a computer capable of representing said electronic file as a three dimensional image.

These and other features, aspects and advantages of the present invention will become better understood with reference to the following description, examples and appended claims.

### DRAWINGS

FIG. 1. Structure of MsbA with Mg.ADP.Vi and LPS. (A) The MsbA homodimer is shown in two orthogonal orientations, within the plane of the bilayer and down the two-fold, respectively. Arrows 1 and 2 indicate the two monomers. The transmembrane domains (TMD) span the lipid bilayer matrix (M) and the nucleotide binding domain (NBD) forms an extensive homodimeric interface within the cytoplasm (C). Two Ra lipopolysaccharide (LPS) molecules (arrow 3) are bound on the periplasmic side (P) of the TMDs. A single ADP molecule (arrow 4) is sequestered within the NBD composite active site.

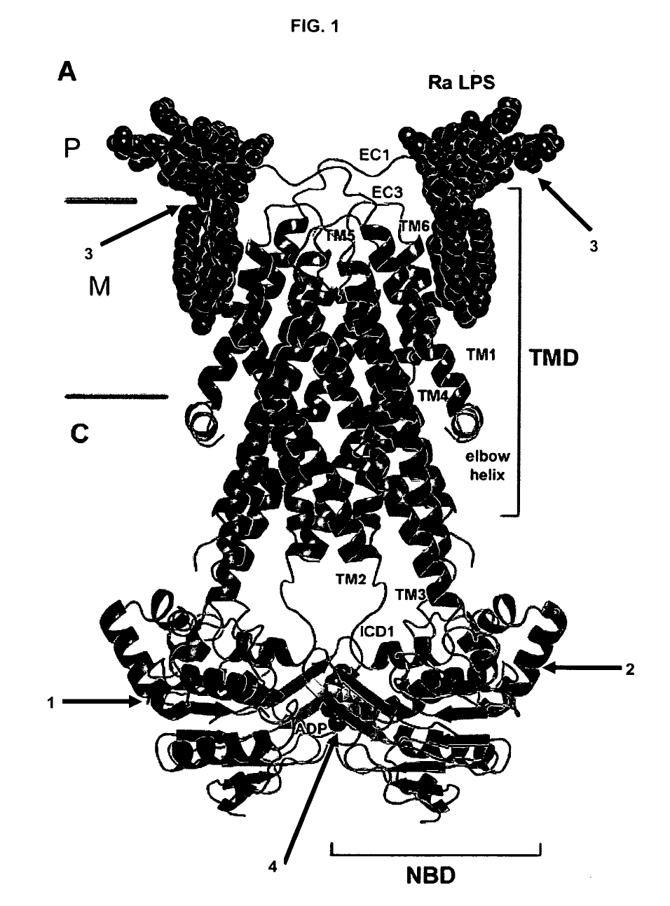

FIG. 2. A different view of FIG. 1; Structure of MsbA with Mg.ADP.Vi and LPS. (B) The MsbA homodimer is shown in two orthogonal orientations, within the plane of the bilayer and down the two-fold, respectively. Arrows 1 and 2 indicate the two monomers. The transmembrane domains (TMD) span the lipid bilayer matrix (M) and the nucleotide binding domain (NBD) forms an extensive homodimeric interface within the cytoplasm (C). Two Ra lipopolysaccharide (LPS) molecules (arrow 3) are bound on the periplasmic side (P) of the TMDs. A single ADP molecule (arrow 4) is sequestered within the NBD composite active site.

FIG. 3. Experimental electron density map (1.0σ) (arrows 5) corresponding to bound LPS.

FIG. 4. Experimental electron density map (1.0σ) (arrows 5) surrounding ADP with anomalous difference maps (4.0σ) corresponding to the 2′-ADP position for Br (arrow 6) and corresponding to the vanadate position (arrow 7; 4.0σ).

FIG. 5. Transmembrane domain rearrangements and specificity. (A) Solvent filled internal chamber (arrow 8) for the closed apo structure of MsbA (arrows 9 and 10 indicate the two monomers).

FIG. 6. (B) Solvent filled internal chamber for MsbA complexed with LPS and ADP (arrows 11 and 12 indicate the two monomers).

FIG. 7. (C) Superimposed TMDs from open apo (arrow 13), closed apo (arrow 14) and post-hydrolysis conformation of MsbA (arrow 15) show the movement of TM5, TM6, and EC3 (as indicated by arrow). The helical bulge near residue Ile²⁵⁷ is indicated by an (*)

FIG. 8. (D) Position of conserved residues shared by MsbA and Pgp (arrow 16) and conserved residues specific to the MsbA subfamily (arrow 17) mapped onto the structure of MsbA (arrow 18). The LPS is indicated by arrow 19.

FIG. 9. The composite catalytic site of the nucleotide binding domains with substrate and conserved sequence motifs. (A) Two bound ATP molecules (arrow 4) are sandwiched between the P loop (arrow 20) and ‘LSGGQ’ (arrow 21) moieties in the composite active site from MJ0796. The Q-loop is indicated by arrow 22.

FIG. 10. (B) Architecture of interacting NBDs from MsbA transition state mimic showing one bound ADP (arrow 4) molecule and one vanadate (arrow 23) per dimer. The nucleotide is bound to the P loop (arrow 24) and disengaged from the conserved ‘LSGGQ’ signature motif from the opposing monomer (arrow 25). The Q-loop is indicated by arrow 22.

FIG. 11. (C) Surface view of single NBD from the apo structure of BtuCD shows no interaction between the L loop and conserved glutamine (Gln⁸⁰) (arrow 26), proposed to coordinate the nucleophilic attacking water (L loop is the ICD1 equivalent, indicated by arrow 27).

FIG. 12. (D) Post hydrolysis intermediate MsbA shows the ICD1 helix (arrow 27) from the TMD interacting with conserved glutamine (Gln⁴²⁴) (arrow 26).

FIG. 13. Model for sequestering polar sugar head group of the LPS in internal chamber of MsbA (only one LPS shown for clarity). (A) LPS initially binds to elbow helix as modeled onto the closed apo structure. (B) Lipid head groups modeled to insert into chamber of apo closed structure. (C) As the transporter undergoes conformational changes related to binding and hydrolysis of ATP, the head group is ‘flipped’ within the polar chamber while the LPS hydrocarbon chains are freely exposed and dragged through the lipid bilayer. Both LPS and MsbA conformations are modeled. (D) LPS is presented to the outer leaflet of the membrane as observed in this structure.

FIG. 14. (A) Functional and structural effects of PSC-833 on MsbA. a, Concentration dependent effect of PSC-833 (PSC) on untreated and doxorubicin stimulated ATPase activity in MsbA.

FIG. 15. (B) PSC mediates doxorubicin retention in ΔacrAB E. coli cells overexpressing MsbA (P<0.05).

FIG. 16. (C) Side view of MsbA homodimer (arrow 28). The transmembrane domain (TMD) spans the lipid bilayer matrix (M), with the intracellular domain (ICD) and the nucleotide binding domain (NBD) within the cytoplasm (C). TMD helices are labeled (TM1-6). Two PSC molecules (arrow 29) are bound at the ICD1/NBD interface and ADP.vanadate (arrow 30) is bound to the ATP active site in one monomer. Highlighted by (*) and (**) are two conserved glycines (Gly141 and Gly94 respectively) in TM2 and TM3 located at the TMD/ICD interface.

FIG. 17. (D) Top view of interacting NBDs down two-fold axis. PSC molecules are bound to each NBD near the A-loop (arrow 31), the ‘RXYD’ loop (arrow 32), ICD1-H2 (arrow 33) and the ‘LSGGQ’ motif (arrow 34).

FIG. 18. (E) Electron density map (1.0σ) corresponding to bound PSC.

FIG. 19. PSC binding site. a, Surface view of PSC binding pocket in stereo. The pocket is formed by conserved NBD elements: A-loop (arrow 31), the ‘RXYD’ loop (arrow 32), ICD1 (arrow 33) and the ‘LSGGQ’ signature motif (arrow 34). PSC is shown as space-filled surface (arrow 35).

FIG. 20. (B) Relationship of chemical representation of PSC to binding pocket elements.

FIG. 21. Sequence alignment of A-loop.

FIG. 22. Sequence alignment of ICD1.

FIG. 23. Sequence alignment of P-loop and the ‘RXYD’ loop of MsbA, P-gp and other ABC transporters showing the sequence conservation of the PSC binding site; especially in the ABCB1/MsbA family.

FIG. 24. Transmembrane domain rearrangements upon PSC binding. a, Lipid-exposed face from the ADP.vanadate.LPS MsbA structure comprised of TM1 (arrow 36), TM6/TM5 (arrow 37) from one monomer and TM2′ (arrow 38) from the other monomer. The periplasmic exposed cleft is formed between TM1 and TM6/TM5. Phe288 is located on TM6 and indicated by arrow 39. b, Lipid-exposed face of MsbA in complex with PSC shows ‘closure’ of TM1-TM6/TM5 cleft with Phe288 buried in the interface as the extracellular loop 2 (EC2) moves away from TM1.

FIG. 25. (A) Proposed mechanism for PSC inhibition of drug transport. The elbow helix/TM1 (left coil throughout), TM6/TM5 (middle coil throughout) and TM2′ (right coil throughout) in the closed apo conformation of MsbA from Vibrio cholera (1). Substrate (ball) initially binds to the inward facing side of membrane. Two grooves could facilitate transport (2). The cis-groove is formed between TM1 and TM6/TM5 from same monomer and the trans-groove is formed between TM6/TM5 and TM2 from opposite monomers. Binding of PSC causes a steric closure of the cis-groove (3), or alternatively, restricts movement of TM6/TM5 which in turn disallows opening of the periplasmic end of the trans-groove (4). Finally, reorientation of TM6/TM5 closes the outward-facing opening of the internal chamber, blocking substrate from exiting the transporter through a pathway between the monomers (5).

FIG. 26. (B) Proposed model of PSC binding to MsbA. ATPase activity shown in the zigzag line and drug transport shown in the line starting from the upper left and proceeding to the lower right. PSC binding alters symmetry and asymmetry in the Intracellular Domains (ICDs), inducing different degrees of Nucleotide Binding Domain (NBD) dimerization. Tight dimerization is essential for nucleotide hydrolysis and is mediated by interaction of opposing ICD1-H2 motifs. (1) In the absence of PSC, MsbA is arranged as a symmetric homodimer and exhibits basal ATPase activity (ICD1 shown in yellow, opposing monomers shown in blue and green). (2) At low concentrations PSC binds one NBD per dimer and promotes asymmetry between the ICD-H2 motifs (yellow and purple), resulting in tight dimerization of the NBDs and stimulation of ATPase activity. (3) At increasing concentrations PSC binds both of the NBDs in the dimer. This restores symmetry, limiting NBD interaction and resulting in ATPase inhibition. (4) At high concentrations PSC binds NBD sites and a transport site in the TMDs. The TMD binding event induces asymmetry in the NBDs via a downward signal through ICD1-H2, resulting in a stimulation of ATPase activity. PSC inhibits drug transport at low concentrations by decoupling ATPase activity from transport in the TMDS, and at high concentrations by competing with drug for the substrate binding sites.

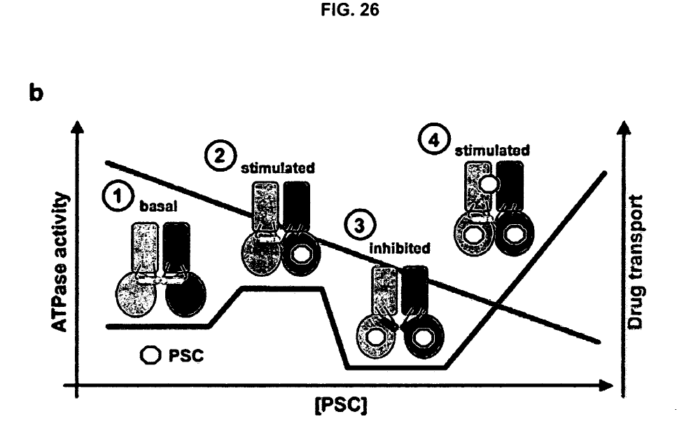

FIG. 27. ATPase control experiments. (A) SDS-PAGE showing greater than 99% purity of detergent solubilized MsbA.

FIG. 28. (B) ATPase specific activity (SA) of MsbA, MsbA plus non-ABC ATPase inhibitor cocktail (1 mM EGTA, 1 mM NaN3, 0.2 mM ouabain), and MsbA plus E coli polar lipids (10 μg/ml).

FIG. 29. (C) Linked enzyme ATPase cocktail alone (third line down), cocktail plus PSC (top line), cocktail plus doxorubicin (second line down), and cocktail plus MsbA (bottom line).

FIG. 30. (D) ATP dependent ATPase activity of MsbA plus/minus 100 μM doxorubicin.

FIG. 31. Sequence alignment of MsbA and P-gp.

### DETAILED DESCRIPTION

The present invention relates to the discovery of the three-dimensional structure of a Transporter-Ligand Complex, models of such three-dimensional structures, a method of structure-based drug design using such structures, the compounds identified by such methods and the use of such compounds in therapeutic compositions. In particular, the present invention relates to a novel crystal of MsbA complexed with ligands LPS and nucleotide, MsbA complexed with ligands PSC-833 and nucleotide, methods of production of such crystal, three dimensional coordinates of such MsbA-ligand complex, a three dimensional structure of the MsbA-ligand complex, and uses of such structure and models to derive other Transporter-Ligand Complex structures and in drug design strategies.

One aspect of the present invention includes a model of a Transporter-Ligand Complex in which the model represents a three dimensional structure of a Transporter-Ligand Complex. Another aspect of the present invention includes the three dimensional structure of a Transporter-Ligand Complex. A three dimensional structure of a Transporter-Ligand Complex substantially conforms with the atomic coordinates represented individually in Table 1 or Table 2. For example, the atomic coordinates represented in Table 2 substantially conform with the atomic coordinates of Table 1, and vice versa. According to the present invention, the use of the term “substantially conforms” refers to at least a portion of a three dimensional structure of a Transporter-Ligand Complex which is sufficiently spatially similar to at least a portion of a specified three dimensional configuration of a particular set of atomic coordinates (e.g., those represented individually by Table 1 or Table 2) to allow the three dimensional structure of a Transporter-Ligand Complex to be modeled or calculated using the particular set of atomic coordinates as a basis for determining the atomic coordinates defining the three dimensional configuration of a Transporter-Ligand Complex.

More particularly, a structure that substantially conforms to a given set of atomic coordinates is a structure wherein at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of such structure has an average root-mean-square deviation (RMSD) of less than about 1.8 Å for the backbone atoms in secondary structure elements in each domain, and in various aspects, less than about 1.25 Å for the backbone atoms in secondary structure elements in each domain, and, in various aspects less than about 1.0 Å, in other aspects less than about 0.75 Å, less than about 0.5 Å, and, less than about 0.25 Å for the backbone atoms in secondary structure elements in each domain. In one aspect of the present invention, a structure that substantially conforms to a given set of atomic coordinates is a structure wherein at least about 75% of such structure has the recited average RMSD value, and in some aspects, at least about 90% of such structure has the recited average RMSD value, and in some aspects, about 100% of such structure has the recited average RMSD value. In particular, the above definition of “substantially conforms” can be extended to include atoms of amino acid side chains. As used herein, the phrase “common amino acid side chains” refers to amino acid side chains that are common to both the structure which substantially conforms to a given set of atomic coordinates and the structure that is actually represented by such atomic coordinates.

In another aspect of the present invention, a three dimensional structure that substantially conforms to a given set of atomic coordinates is a structure wherein at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the common amino acid side chains have an average RMSD of less than about 1.8 Å, and in various aspects, less than about 1.25 Å, and, in other aspects, less than about 1.0 Å, less than about 0.75 Å, less than about 0.5 Å, and less than about 0.25 Å. In one aspect of the present invention, a structure that substantially conforms to a given set of atomic coordinates is a structure wherein at least about 75% of the common amino acid side chains have the recited average RMSD value, and in some aspects, at least about 90% of the common amino acid side chains have the recited average RMSD value, and in some aspects, about 100% of the common amino acid side chains have the recited average RMSD value.

A three dimensional structure of a Transporter-Ligand Complex which substantially conforms to a specified set of atomic coordinates can be modeled by a suitable modeling computer program such as MODELER (A. Sali and T. L. Blundell, J. Mol. Biol., vol. 234:779-815, 1993 as implemented in the Insight II software package Insight II, available from Accelerys (San Diego, Calif.)) and those software packages listed in the Examples, using information, for example, derived from the following data: (1) the amino acid sequence of the Transporter-Ligand Complex; (2) the amino acid sequence of the related portion(s) of the protein represented by the specified set of atomic coordinates having a three dimensional configuration; and, (3) the atomic coordinates of the specified three dimensional configuration. A three dimensional structure of a Transporter-Ligand Complex which substantially conforms to a specified set of atomic coordinates can also be calculated by a method such as molecular replacement, which is described in detail below.

A suitable three dimensional structure of the Transporter-Ligand Complex for use in modeling or calculating the three dimensional structure of another Transporter-Ligand Complex comprises the set of atomic coordinates represented individually in Table 1 or Table 2. The set of three dimensional coordinates set forth in Tables 1 and 2 are represented in standard Protein Data Bank format. According to the present invention, a transporter-ligand complex has a three dimensional structure which substantially conforms to the set of atomic coordinates individually represented by Table 1 or Table 2. As used herein, a three dimensional structure can also be a most probable, or significant, fit with a set of atomic coordinates. According to the present invention, a most probable or significant fit refers to the fit that a particular Transporter-Ligand Complex has with a set of atomic coordinates derived from that particular Transporter-Ligand Complex. Such atomic coordinates can be derived, for example, from the crystal structure of the protein such as the coordinates determined for the Transporter-Ligand Complex structure provided herein, or from a model of the structure of the protein. For example, the three dimensional structure of a dimeric protein, including a naturally occurring or recombinantly produced ABC Transporter protein, substantially conforms to and is a most probable fit, or significant fit, with the atomic coordinates of Table 1 or Table 2 individually. The three dimensional crystal structure of the Transporter-Ligand Complex may individually comprise the atomic coordinates of Table 1 or Table 2. Also as an example, the three dimensional structure of another Transporter-Ligand Complex would be understood by one of skill in the art to substantially conform to the atomic coordinates of Table 1 or Table 2 individually. This definition can be applied to the other ABC Transporter proteins in a similar manner.

For example, the structure of MsbA establishes the general architecture of the MDR-ABC transporter family. Accordingly, in some configurations, ABC Transporter protein sequence homology across both prokaryotes and eukaryotes is used as a basis to predict the structure of such transporters, in particular the structure for such transporter-ligands binding sites and other conserved regions.

In various aspects of the present invention, a structure of a Transporter-Ligand Complex substantially conforms to the atomic coordinates represented individually in Table 1 or Table 2. Such values as listed in Tables 1 and 2 can be interpreted by one of skill in the art. In other aspects, a three dimensional structure of a Transporter-Ligand Complex substantially conforms to the three dimensional coordinates represented in Tables 1 and 2. In other aspects, a three dimensional structure of a Transporter-Ligand Complex is a most probable fit with the three dimensional coordinates represented individually in Table 1 or Table 2. Methods to determine a substantially conforming and probable fit are within the expertise of skill in the art and are described herein in the Examples section.

A Transporter-Ligand Complex that has a three dimensional structure which substantially conforms to the atomic coordinates represented individually by Table 1 or Table 2 includes an ABC Transporter protein having an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence of a prokaryotic or eukaryotic MsbA protein, in particular an amino acid sequence having one of SEQ ID NOs: 17-31, across the full-length of the MsbA sequence. A sequence alignment program such as BLAST (available from the National Institutes of Health Internet web site http://www.ncbi.nim.nih.gov/BLAST) may be used by one of skill in the art to compare sequences of an ABC Transporter to MsbA.

A three dimensional structure of any Transporter-Ligand Complex can be modeled using methods generally known in the art based on information obtained from analysis of a Transporter-Ligand Complex crystal, and from other Transporter-Ligand Complex structures which are derived from a Transporter-Ligand Complex crystal. The Examples section below discloses the production of a Transporter-Ligand Complex crystal, in particular MsbA dimers complexed with LPS and nucleotide, and a model of a Transporter-Ligand Complex, in particular the three dimensional structure of MsbA dimers complexed with LPS and nucleotide, using methods generally known in the art based on the information obtained from analysis of a Transporter-Ligand Complex crystal. An aspect of the present invention comprises using the three dimensional structure of a crystalline Transporter-Ligand Complex to derive the three dimensional structure of another Transporter-Ligand Complex. Therefore, the crystalline MsbA dimers complexed with LPS and nucleotide and the three dimensional structure of MsbA dimers complexed with LPS and nucleotide permits one of ordinary skill in the art to now derive the three dimensional structure, and models thereof, of any Transporter-Ligand Complex. The derivation of the structure of any Transporter-Ligand Complex can now be achieved even in the absence of having crystal structure data for such other Transporter-Ligand Complexes, and when the crystal structure of another Transporter-Ligand Complex is available, the modeling of the three dimensional structure of the new Transporter-Ligand Complex can be refined using the knowledge already gained from the Transporter-Ligand Complex structure.

In some configurations of the present teachings, the absence of crystal structure data for other Transporter-Ligand Complexes, the three dimensional structures of other Transporter-Ligand Complexes can be modeled, taking into account differences in the amino acid sequence of the other Transporter-Ligand Complex. Moreover, the present invention allows for structure based drug design of compounds which affect the activity of virtually any ABC Transporter, and particularly, of MsbA.

One aspect of the present invention includes a three dimensional structure of a Transporter-Ligand Complex, in which the atomic coordinates of the Transporter-Ligand Complex are generated by the method comprising: (a) providing an ABC Transporter complexed with a ligand in crystalline form; (b) generating an electron-density map of the crystalline ABC Transporter complexed with the ligand; and (c) analyzing the electron-density map to produce the atomic coordinates. For example, the structure of MsbA from Salmonella typhimurium in complex with ADP.Vi, Mg²⁺ and Ra lipopolysaccharide (Ra LPS) is provided herein. In another example, the structure of MsbA from Salmonella typhimurium in complex with PSC-833 and nucleotide is is provided.

As used herein, PSC-833 refers to [3′-Desoxy-3′-oxo-MeBmt]¹-[Val]²-Ciclosporin. That compound and its activity is represented in U.S. Pat. No. 5,525,590 which is incorporated herein by reference in its entirety. Other compounds related to PSC-833 and having similar activity are also contemplated to be useful in the present invention and may be referred to “PSC-833 related compounds” and as a “cyclosporin D analogue.” Such related compounds are provided in U.S. Pat. No. 5,525,590.

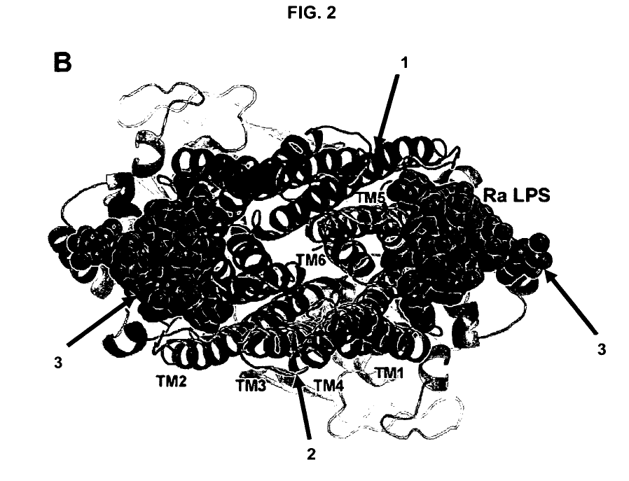

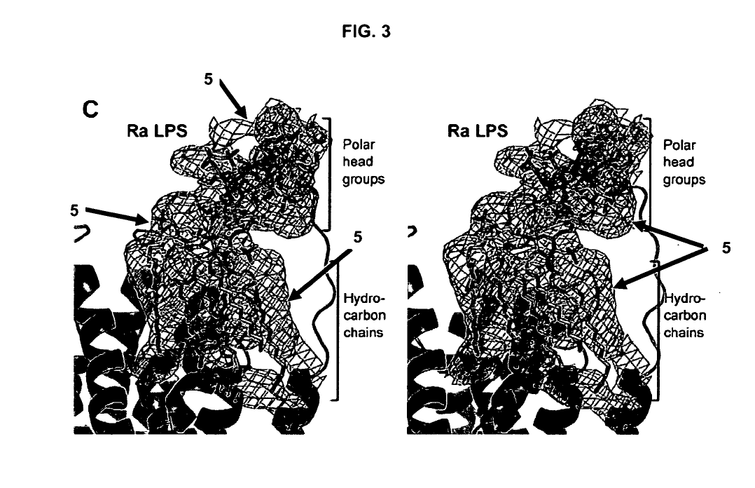

Crystals of MsbA in complex with Mg.ADP.Vi and Ra LPS were grown using detergent solubilized protein incubated with Ra LPS purified from S. typhimurium. ATP, Mg²⁺, and boiled sodium orth-vanadate were added to favor the transition state conformation prior to crystallization. Mass spectrometry on washed crystals indicated the presence of Ra LPS, nucleotide, and vanadate (data not shown). The structure was determined by single wavelength anomalous dispersion (SAD) and the electron density maps were improved by using non-crystallographic symmetry averaging to a resolution of 4.2 Å (Table 3). The asymmetric unit revealed two dimers of MsbA with clear electron density corresponding to nucleotide and Ra LPS. The TMDs in each dimer exhibits a 30° torque relative to the molecular two fold axis and an extensive interdigitation of the helices (FIGS. 2 and 3). A chemical model with good geometry was built with R_{cryst} of 28% and R_{free} of 33% (Table 3). Crystals of MsbA in complex with PSC-833 and nucleotide were prepared according to Example 2 below.

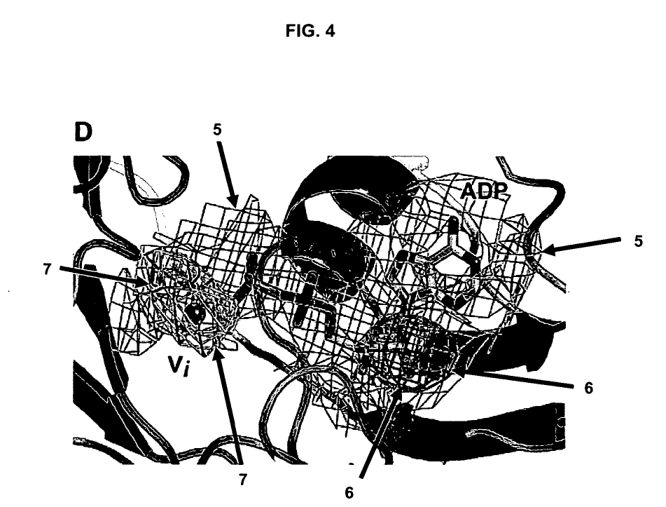

In the structure of MsbA from S. typhimurium, each dimer contains two bound LPS molecules located at the protein-membrane interface on the outer membrane leaflet side with the sugar head groups roughly parallel to the axis of the elbow helix (residues 10-24) (FIG. 3). Electron density was observed for only one nucleotide per dimer in the active site position (FIG. 4). To confirm the positioning of the ADP and vanadate at this resolution, ATP was substituted with a 2′-brominated ATP analog and anomalous diffraction data was collected at the bromine edge (λ=0.9198 Å). Anomalous difference Fourier synthesis using experimental protein phases yielded only one bromine peak (˜5σ) per NDB dimer corresponding to the observed electron density for the nucleotide at the 2′ position (FIG. 4). Anomalous diffraction data were then collected at the vanadium edge (λ=5.4651 Å) and as with the bromine anomalous maps, the vanadate maps were phased and a single peak (˜4σ) was observed corresponding the predicted position for coordinated vanadate based on the Mg.ADP.Vi structure of Myosin. Neither the coordinated oxygen atoms of the vanadate ion nor Mg²⁺ are included in the model of the present invention.

According to the present invention, a three dimensional structure of the MsbA protein complexed with LPS and nucleotide can be used to derive a model of the three dimensional structure of another Transporter-Ligand Complex (i.e., a structure to be modeled). As used herein, a “structure” of a protein refers to the components and the manner of arrangement of the components to constitute the protein. As used herein, the term “model” refers to a representation in a tangible medium of the three dimensional structure of a protein, polypeptide or peptide. For example, a model can be a representation of the three dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Accelrys, Inc. (San Diego, Calif.). The phrase “providing a picture of the model” refers to the ability to generate a “hard copy” of the model. Hard copies include both motion and still pictures. Computer screen images and pictures of the model can be visualized in a number of formats including space-filling representations, α-carbon traces, ribbon diagrams and electron density maps.

Suitable target Transporter-Ligand Complex structures to model using a method of the present invention include any ABC Transporter protein, polypeptide or peptide, including monomers, dimers and multimers of an ABC Transporter protein, that is substantially structurally related to an MsbA protein complexed with LPS and nucleotide protein, i.e., human MDR1/ABCB1 (e.g., SEQ ID NO: 23), or an ABC Transporter protein that is substantially structurally related to an MsbA protein complexed with PSC-833 or related compounds. In various embodiments, a target Transporter-Ligand Complex structure that is substantially structurally related to an ABC Transporter protein includes a target Transporter-Ligand Complex structure having an amino acid sequence that is at least about 25%, 26%, 27%, 28%, 29%, 30%, 31%, 32%, 33%, 34%, 35%, 36%, 37%, 38%, 38%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence of a prokaryotic or eukaryotic MsbA protein, in particular an amino acid sequence having one of SEQ ID NOs: 17-31, across the full-length of the MsbA sequence when using, for example, a sequence alignment program such as BLAST (supra). In various aspects of the present invention, target Transporter-Ligand Complex structures to model include proteins comprising amino acid sequences that are at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence having one of SEQ ID NOs: 17-31 when comparing suitable regions of the sequence, such as the amino acid sequence for an NDB or cationic hydrophobic molecule binding site of any one of the amino acid sequences, when using an alignment program such as BLAST (supra) to align the amino acid sequences.

According to the present invention, a structure can be modeled using techniques generally described by, for example, Sali, Current Opinions in Biotechnology, vol. 6, pp. 437-451, 1995, and algorithms can be implemented in program packages such as Insight II, available from Accelerys (San Diego, Calif.). Use of Insight II HOMOLOGY requires an ailignment of an amino acid sequence of a known structure having a known three dimensional structure with an amino acid sequence of a target structure to be modeled. The alignment can be a pairwise alignment or a multiple sequence alignment including other related sequences (for example, using the method generally described by Rost, Meth. Enzymol., vol. 266, pp. 525-539, 1996) to improve accuracy. Structurally conserved regions can be identified by comparing related structural features, or by examining the degree of sequence homology between the known structure and the target structure. Certain coordinates for the target structure are assigned using known structures from the known structure. Coordinates for other regions of the target structure can be generated from fragments obtained from known structures such as those found in the Protein Data Bank. Conformation of side chains of the target structure can be assigned with reference to what is sterically allowable and using a library of rotamers and their frequency of occurrence (as generally described in Ponder and Richards, J. Mol. Biol., vol.193, pp. 775-791, 1987). The resulting model of the target structure, can be refined by molecular mechanics to ensure that the model is chemically and conformationally reasonable.

Accordingly, one embodiment of the present invention is a method to derive a model of the three dimensional structure of a target Transporter-Ligand Complex structure, the method comprising the steps of: (a) providing an amino acid sequence of a Transporter-Ligand Complex and an amino acid sequence of a target ligand-complexed ABC Transporter; (b) identifying structurally conserved regions shared between the Transporter-Ligand Complex amino acid sequence and the target ligand-complexed ABC Transporter amino acid sequence; (c) determining atomic coordinates for the target ligand-complexed ABC Transporter by assigning said structurally conserved regions of the target ligand-complexed ABC Transporter to a three dimensional structure using a three dimensional structure of a Transporter-Ligand Complex based on atomic coordinates that substantially conform to the atomic coordinates represented individually in Table 1 or Table 2, to derive a model of the three dimensional structure of the target ligand-complexed ABC Transporter amino acid sequence. A model according to the present invention has been previously described herein. In one aspect, the model comprises a computer model. The method can further comprise the step of electronically simulating the structural assignments to derive a computer model of the three dimensional structure of the target ligand-complexed ABC Transporter amino acid sequence.

Another embodiment of the present invention is a method to derive a computer model of the three dimensional structure of a target ligand-complexed ABC Transporter structure for which a crystal has been produced (referred to herein as a “crystallized target structure”). A suitable method to produce such a model includes the method comprising molecular replacement. Methods of molecular replacement are generally known by those of skill in the art and are performed in a software program including, for example, X-PLOR available from Accelerys (San Diego, Calif.). In various aspects, a crystallized target ligand-complexed ABC Transporter structure useful in a method of molecular replacement according to the present invention has an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the search structure (e.g., MsbA and MDR1), when the two amino acid sequences are compared using an alignment program such as BLAST (supra). A suitable search structure of the present invention includes an Transporter-Ligand Complex having a three dimensional structure that substantially conforms with the atomic coordinates listed individually in Table 1 or Table 2.

Another aspect of the present invention is a method to determine a three dimensional structure of a target Transporter-Ligand Complex structure, in which the three dimensional structure of the target Transporter-Ligand Complex structure is not known. Such a method is useful for identifying structures that are related to the three dimensional structure of a Transporter-Ligand Complex based only on the three dimensional structure of the target structure. For example, the present method enables identification of structures that do not have high amino acid identity with an MsbA protein but which share three dimensional structure similarities of a ligand-complexed MsbA. In various aspects of the present invention, a method to determine a three dimensional structure of a target Transporter-Ligand Complex structure comprises: (a) providing an amino acid sequence of a target structure, wherein the three dimensional structure of the target structure is not known; (b) analyzing the pattern of folding of the amino acid sequence in a three dimensional conformation by fold recognition; and (c) comparing the pattern of folding of the target structure amino acid sequence with the three dimensional structure of a Transporter-Ligand Complex to determine the three dimensional structure of the target structure, wherein the three dimensional structure of the Transporter-Ligand Complex substantially conforms to the atomic coordinates represented individually in Table 1 or Table 2. For example, methods of fold recognition can include the methods generally described in Jones, Curr. Opinion Struc. Biol., vol. 7, pp. 377-387, 1997. Such folding can be analyzed based on hydrophobic and/or hydrophilic properties of a target structure.

One aspect of the present invention includes a three dimensional computer image of the three dimensional structure of a Transporter-Ligand Complex. In one aspect, a computer image is created to a structure which substantially conforms with the three dimensional coordinates listed individually in Table 1 or Table 2. A computer image of the present invention can be produced using any suitable software program, including, but not limited to, Pymol available from DeLano Scientific, LLC (South San Francisco, Calif.). Suitable computer hardware useful for producing an image of the present invention are known to those of skill in the art.

Another aspect of the present invention relates to a computer-readable medium encoded with a set of three dimensional coordinates represented individually in Table 1 or Table 2, wherein, using a graphical display software program, the three dimensional coordinates create an electronic file that can be visualized on a computer capable of representing said electronic file as a three dimensional image. Yet another aspect of the present invention relates to a computer-readable medium encoded with a set of three dimensional coordinates of a three dimensional structure which substantially conforms to the three dimensional coordinates represented individually in Table 1 or Table 2, wherein, using a graphical display software program, the set of three dimensional coordinates create an electronic file that can be visualized on a computer capable of representing said electronic file as a three dimensional image.

The present invention also includes a three dimensional model of the three dimensional structure of a target structure, such a three dimensional model being produced by the method comprising: (a) providing an amino acid sequences of an ABC Transporter comprised by a Transporter-Ligand Complex and an amino acid sequence of a target Transporter-Ligand Complex structure; (b) identifying structurally conserved regions shared between the ABC Transporter amino acid sequence and the amino acid sequence comprised by the target Transporter-Ligand Complex structure; (c) determining atomic coordinates for the target Transporter-Ligand Complex by assigning the structurally conserved regions of the target Transporter-Ligand Complex to a three dimensional structure using a three dimensional structure of the ABC Transporter comprised by a Transporter-Ligand Complex based on atomic coordinates that substantially conform to the atomic coordinates represented individually in Table 1 or Table 2 to derive a model of the three dimensional structure of the target Transporter-Ligand Complex. In one aspect, the model comprises a computer model.

Any isolated ABC Transporter protein can be used with the methods of the present invention. An isolated ABC Transporter protein can be isolated from its natural milieu or produced using recombinant DNA technology (e.g., polymerase chain reaction (PCR) amplification, cloning) or chemical synthesis. To produce recombinant ABC Transporter protein, a nucleic acid molecule encoding ABC Transporter protein can be inserted into any vector capable of delivering the nucleic acid molecule into a host cell. A nucleic acid molecule of the present invention can encode any portion of an ABC Transporter protein, in various aspects a full-length ABC Transporter protein, and in various aspects a soluble form of ABC Transporter protein (i.e., a form of ABC Transporter protein capable of being secreted by a cell that produces such protein). A suitable nucleic acid molecule to include in a recombinant vector, and particularly in a recombinant molecule, includes a nucleic acid molecule encoding a protein having the amino acid sequence represented by individual sequences or any combination of SEQ ID NOs: 17-31.

A recombinant vector can be either RNA or DNA, either prokaryotic or eukaryotic, and typically is a virus or a plasmid. In various aspects, a nucleic acid molecule encoding an ABC Transporter protein is inserted into a vector comprising an expression vector to form a recombinant molecule. As used herein, an expression vector is a DNA or RNA vector that is capable of transforming a host cell and of affecting expression of a specified nucleic acid molecule. Expression vectors of the present invention include any vectors that function (i.e., direct gene expression) in recombinant cells of the present invention, including in bacterial, fungal, endoparasite, insect, other animal, and plant cells.

An expression vector can be transformed into any suitable host cell to form a recombinant cell. A suitable host cell includes any cell capable of expressing a nucleic acid molecule inserted into the expression vector. For example, a prokaryotic expression vector can be transformed into a bacterial host cell. One method to isolate ABC Transporter protein useful for producing ligand-complexed ABC Transporter crystals includes recovery of recombinant proteins from cell cultures of recombinant cells expressing such ABC Transporter protein.

ABC Transporter proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization. In various aspects of the present invention, an ABC Transporter protein is purified in such a manner that the protein is purified sufficiently for formation of crystals useful for obtaining information related to the three dimensional structure of an Transporter-Ligand Complex. In some aspects, a composition of ABC Transporter protein is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% pure.

Another embodiment of the present invention includes a composition comprising a Transporter-Ligand Complex in a crystalline form (i.e., Transporter-Ligand Complex crystals). As used herein, the terms “crystalline Transporter-Ligand Complex” and “Transporter-Ligand Complex crystal” both refer to crystallized a Transporter-Ligand Complex and are intended to be used interchangeably. In various aspects of the present invention, a crystalline Transporter-Ligand Complex is produced using the crystal formation method described in the Examples.

In particular, the present invention includes a composition comprising MsbA complexed with LPS and nucleotide or MsbA complexed with PSC-833 and nucleotide in a crystalline form (i.e., ligand-complexed MsbA crystals). As used herein, the terms “crystalline ligand-complexed MsbA” and “ligand-complexed MsbA crystal” both refer to crystallized MsbA complexed with LPS and nucleotide or MsbA complexed with PSC-833 and nucleotide and are intended to be used interchangeably. In various aspects of the present invention, a crystal ligand-complexed MsbA is produced using the crystal formation method described in the Examples. In some aspects, a composition of the present invention includes ligand-complexed (e.g., LPS and nucleotide complexed) MsbA molecules arranged in a crystalline manner in a space group C2, so as to form a unit cell of dimensions a=271.4 Å, b=122.0 Å, c=121.7 Å, α=90°, β=121.7 °, γ=90°. In other aspects, the ligand-complexed (e.g., PSC-833 and nucleotide complexed) MsbA molecules arranged in a crystalline manner in a space group C2, so as to form a unit cell of dimensions a=268.2 Å, b=121.4 Å, c=176.2 Å, and β=121.2°. A suitable crystal of the present invention provides X-ray diffraction data for determination of atomic coordinates of the ligand-complexed MsbA to a resolution of about 4.2 Å, and in some aspects about 3.0 Å, and in other aspects at about 1.5 Å.

According to an aspect of the present invention, crystalline Transporter-Ligand Complex can be used to determine the ability of a compound of the present invention to bind to an ABC Transporter in a manner predicted by a structure based drug design method of the present invention. In various aspects of the present invention, a Transporter-Ligand Complex crystal is soaked in a solution containing a chemical compound of the present invention. Binding of the chemical compound to the crystal is then determined by methods standard in the art.

One aspect of the present invention is a therapeutic composition. A therapeutic composition of the present invention comprises one or more therapeutic compounds. In one aspect, a therapeutic composition is provided that is capable of reducing MDR that involves an ABC Transporter. For example, a therapeutic composition of the present invention can inhibit (i.e., prevent, block) binding of an ABC Transporter on a cell having an ABC Transporter (e.g., prokaryotic bacteria and eukaryotic cells) to a, e.g., cationic hydrophobic molecule by interfering with a cationic hydrophobic binding site of an ABC Transporter. As used herein, the term “binding site” refers to the region of a molecule (e.g., a cationic hydrophobic molecule) to which another molecule specifically binds. In one aspect of the present invention, a method is provided for inhibiting MDR in a subject comprising administering to the subject in need thereof a therapeutically effective amount of a therapeutic composition of the present invention.

Suitable inhibitory compounds of the present invention are compounds that interact directly with an ABC Transporter protein, and in various aspects an MsbA protein, thereby inhibiting the binding of an ABC Transporter ligand, e.g., LPS and nucleotide, to an ABC Transporter, by blocking the ligand binding site of an ABC Transporter (referred to herein as substrate analogs). An ABC Transporter substrate analog refers to a compound that interacts with (e.g., binds to, associates with, modifies) the binding site of an ABC Transporter. An ABC Transporter substrate analog can, for example, comprise a chemical compound that mimics the Ra LPS portion of LPS, or that binds specifically to the NBS, LPS or other ligand binding site of an ABC Transporter. In one non limiting example, PSC-833 can be an inhibitor compound.

According to the present invention, suitable therapeutic compounds of the present invention include peptides or other organic molecules, and inorganic molecules. Suitable organic molecules include small organic molecules. In various aspects, a therapeutic compound of the present invention is not harmful (e.g., toxic) to an animal when such compound is administered to an animal. Peptides refer to a class of compounds that is small in molecular weight and yields two or more amino acids upon hydrolysis. A polypeptide is comprised of two or more peptides. As used herein, a protein is comprised of one or more polypeptides. Suitable therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations.

Therapeutic compounds of the present invention can be designed using structure based drug design. Structure based drug design refers to the use of computer simulation to predict a conformation of a peptide, polypeptide, protein, or conformational interaction between a peptide or polypeptide, and a therapeutic compound. In the present teachings, knowledge of the three dimensional structure of the LPS binding site or NBS of an ABC Transporter provide one of skill in the art the ability to design a therapeutic compound that binds to ABC Transporters, is stable and results in inhibition of a biological response, such as cationic hydrophobic molecule extrusion from a cell having an ABC Transporter. For example, knowledge of the three dimensional structure of the LPS binding site or NBS of an ABC Transporter provides to a skilled artisan the ability to design an analog of an LPS or a nucleotide which can function as a substrate or ligand of an ABC transporter. In another example, knowledge of the three dimensional structure of the PSC-833 or related compound binding site of an ABC Transporter provides to a skilled artisan the ability to design an analog of PSC-833 or related compound which can function as a substrate or ligand of an ABC transporter.

Suitable structures and models useful for structure-based drug design are disclosed herein. Models of target structures to use in a method of structure-based drug design include models produced by any modeling method disclosed herein, such as, for example, molecular replacement and fold recognition related methods. In some aspects of the present invention, structure based drug design can be applied to a structure of MsbA in complex with LPS and nucleotide, and to a model of a target ABC Transporter structure.

One embodiment of the present invention is a method for designing a drug which interferes with an activity of an ABC transporter. In various configurations, the method comprises providing a three-dimensional structure of a Transporter Ligand Complex comprising the ABC transporter and at least one ligand of the transporter; and designing a chemical compound which is predicted to bind to the ABC transporter. The designing can comprise using physical models, such as, for example, ball-and-stick representations of atoms and bonds, or on a digital computer equipped with molecular modeling software. In some configurations, these methods can further include synthesizing the chemical compound, and evaluating the chemical compound for ability to interfere with an activity of the ABC transporter.

Suitable three dimensional structures of a Transporter-Ligand Complex and models to use with the present method are disclosed herein. According to the present invention, designing a compound can include creating a new chemical compound or searching databases of libraries of known compounds (e.g., a compound listed in a computational screening database containing three dimensional structures of known compounds). Designing can also include simulating chemical compounds having substitute moieties at certain structural features. In some configurations, designing can include selecting a chemical compound based on a known function of the compound, e.g., PSC-833. In some configurations designing can comprise computational screening of one or more databases of compounds in which three dimensional structures of the compounds are known. In these configurations, a candidate compound can be interacted virtually (e.g., docked, aligned, matched, interfaced) with the three dimensional structure of a Transporter-Ligand Complex by computer equipped with software such as, for example, the AutoDock software package, (The Scripps Research Institute, La Jolla, Calif.) or described by Humblet and Dunbar, Animal Reports in Medicinal Chemistry, vol. 28, pp. 275-283, 1993, M Venuti, ed., Academic Press. Methods for synthesizing candidate chemical compounds are known to those of skill in the art.

Various other methods of structure-based drug design are disclosed in references such as Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional structures and small fragment probes, followed by linking together of favorable probe sites.

In one aspect, a chemical compound of the present invention that binds to the LPS binding site or NBS of a Transporter-Ligand Complex can be a chemical compound having chemical and/or stereochemical complementarity with an ABC Transporter, e.g., an MsbA, or MsbA ligand, such as, for example, LPS, PSC-833 or related compounds, or nucleotide. In some configurations, a chemical compound that binds to the LPS binding site or NBS of an ABC Transporter can associate with an affinity of at least about 10⁻⁶ M, at least about 10⁻⁷ M, or at least about 10⁻⁸ M.

Several sites of ABC Transporters can be targets for structure based drug design. These sites include, in non-limiting example residues which contact LPS (e.g., TM1: 37-44, TM2: 57-78, and TM6: 277-291); extracellular loops (e.g., EC1: 49-68, EC2: 160-168 EC3: 270-278); intercellular Domain 1 (ICD1: 92-142); and CMNP-specific interactions (elbow helix: 10-22, TM1: 26-37, TM3: 143-153, TM4: 180-187, TM5: 251-256, TM6: 291-303). In addition, residues which contact PSC-833 are also included: LSGGQ, A-loop, RXYD, ICD1 and a combination thereof. The LSGGQ site can comprise L481. The A-loop site can comprise at least one amino acid selected from the group consisting of F349, Y351, P352, G353, R354 and E355. The RXYD site can comprise at least one amino acid selected from the group consisting of R391, F392, Y393, D394, 1395 and D396. The ICD1 site can comprise at least one amino acid selected from the group consisting of M108 and F115.

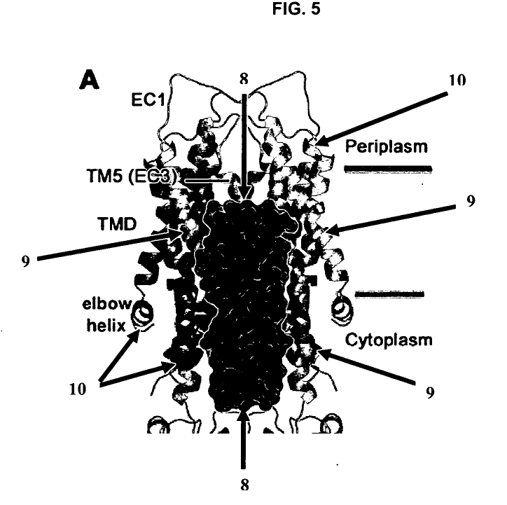

Prior structural analysis has shown MsbA in the absence of nucleotide comprises open and closed conformations. In the open conformation, the two TMDs interact at the extracellular ends of the membrane-spanning helices forming an inverted “V” shaped molecule with NBDs distant from each other. In the closed apo conformation, both the TMDs and the NBDs are closely packed. In addition, a large internal chamber accessible from the cytoplasm is formed between the interacting TMDs (FIG. 5). The two structures indicate a highly dynamic sampling of conformational space by the protein in the absence of nucleotide with respect to the TMD interactions, lipid bilayer arrangements, and the folding of the NBDs. Prior electron paramagnetic resonance (EPR) studies also indicate that movements in the TMD are dramatic.

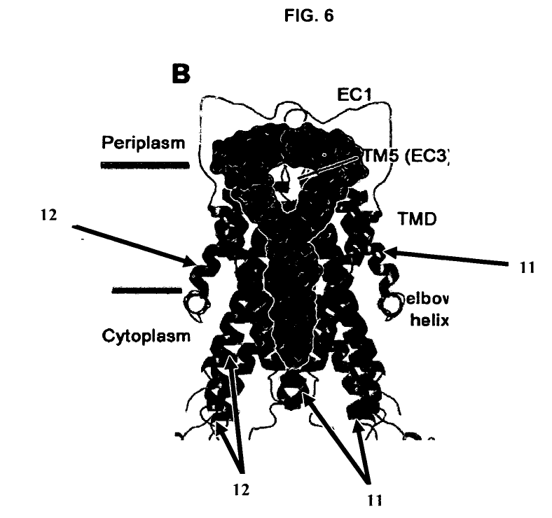

Changes in the TMD interactions provide insight into the pathway of substrate efflux. In comparison to the apo closed conformation of MsbA, this structure exhibits a large rigid body rotation and translation that result in a ˜15 Å opening towards the periplasmic ends and a ˜15 Å closing of the NBD associated intracellular domain helices (ICD1; residues 111-121) allowing accessibility to an internal chamber from the periplasm but not from the cytoplasm (FIG. 6). The third extracellular loops (EC3) mediate the internal contact between the two monomers while placing the periplasmic ends of the TM5 helices close together. This causes the periplasmic opening of the internal chamber to be pinched and divides the opening of the cavity into two lobes adjacent to TM6, coincident with drug binding sites observed for LmrA (30) and human P-gp (31). In the MsbA structure of the present invention, TM5 forms extensive intermolecular interactions with TM2 and TM3. As the ICD1 is formed between TM2 and TM3, this interaction provides a pathway for transmitting conformational changes caused by ATP hydrolysis to the substrate binding sites.

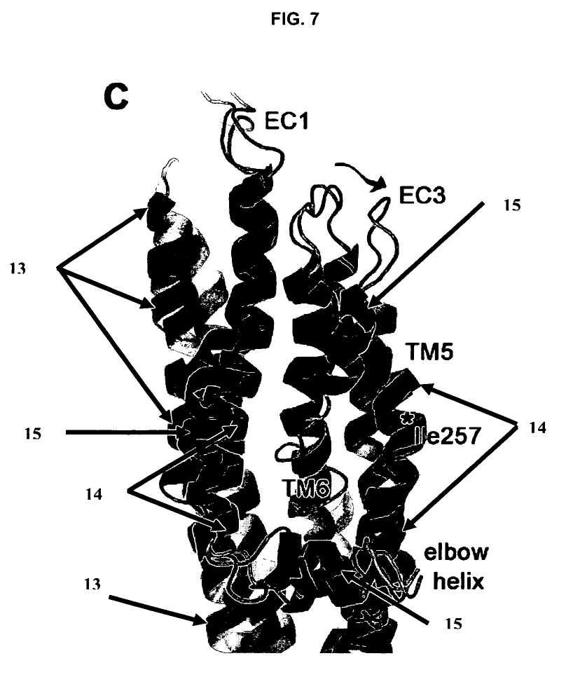

Besides affecting the interactions between TMDs, substrate binding and ATP hydrolysis also drive changes in the intermolecular helical packing of the TMDs. While the TM14 helices are arranged in a similar overall architecture in comparison to both the open and closed apo conformations, TM5 and TM6 reveal significant rearrangements (RMSD on Cα's of 2.1 Å (open) and 1.9 Å (closed)) (FIG. 7). In both apo structures, a conserved residue (Ile257—S. typhimurium MsbA ) is located near a helical bulge that characterizes TM5. In this structure, this region has moved towards the interior of the TMD facilitating intermolecular contacts within the dimer. Similarly, the periplasmic end of TM6 has moved out of the interior contributing to a greater than 7 Å shift in EC3 between the apo and transition state conformation. The consequence of these intermolecular movements is a concerted movement of TM1, TM6, and the elbow helix towards the cell membrane.

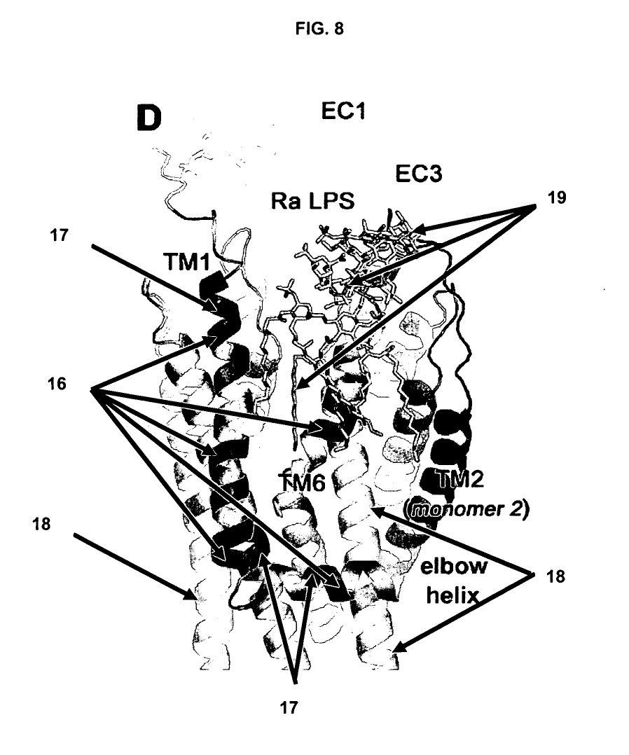

A critical question is how substrate specificity is shared within the subfamily of MDR ABC flippases. In this structure, LPS is bound on the outer membrane leaflet side of MsbA, forming extensive contacts with TM1 and TM6 from one monomer and with TM2 from the other monomer (FIG. 8). The hydrocarbon chains of the LPS interact with apolar residues on this interface and the head groups interact with polar residues near the periplasmic side of the TMD, which includes the first extracellular loop, EC1 (residues 54 to 68). Furthermore, several conserved residues between MsbA and P-gp map to the binding interface of the LPS indicating that this region can be a general binding site for other hydrophobic compounds. These residues are localized on the elbow helix, TM1, and TM6. Conserved residues specific to the MsbA subfamily also map to the EC1 loop, which interacts with the LPS sugar head groups from opposing monomers, and the elbow helix (FIG. 8).

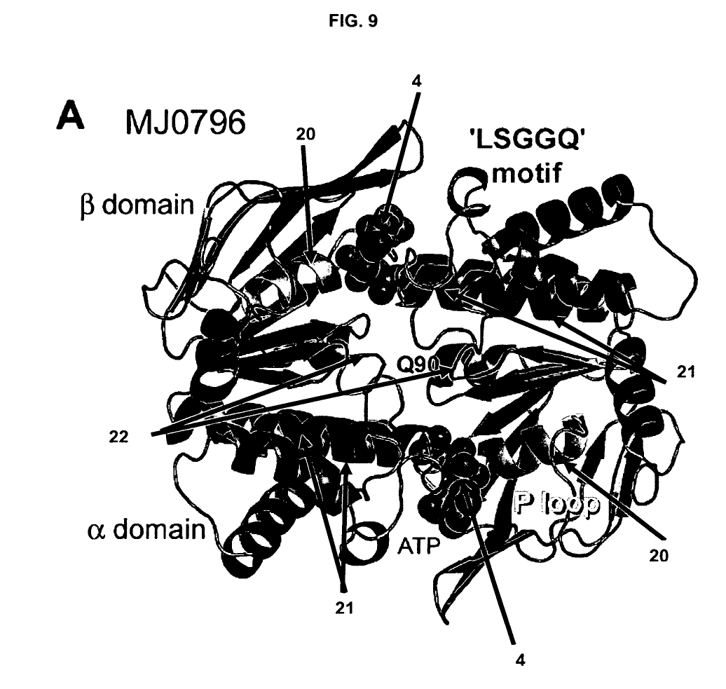

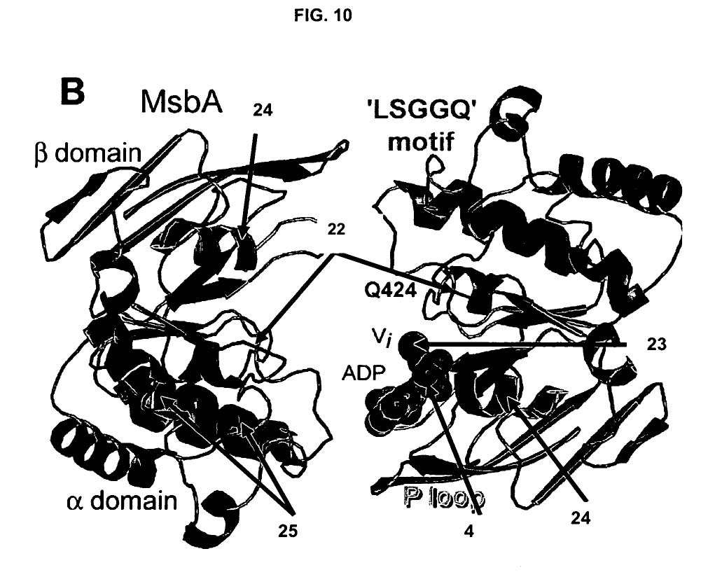

The architecture of the NBDs in the complex resembles the dimer sandwich structures of the ABC transporters BtuCD and MJ0796 with the P-loop and the ‘LSGGQ’ signature motif anchoring two subdomains of the NBD (FIGS. 9 and 10). In the ATP bound form of the MJ0796 NBD dimer, the two motifs from opposing NBDs align to orient the bound ATP molecules and form a composite active site. In the complex, nucleotide is in only one of the active sites. In addition, the signature motif from opposing NBDs disengages from the P-loop and bound nucleotide. This can be interpreted to imply a post hydrolysis conformation and that the nucleotide from the empty binding site has already dissociated.

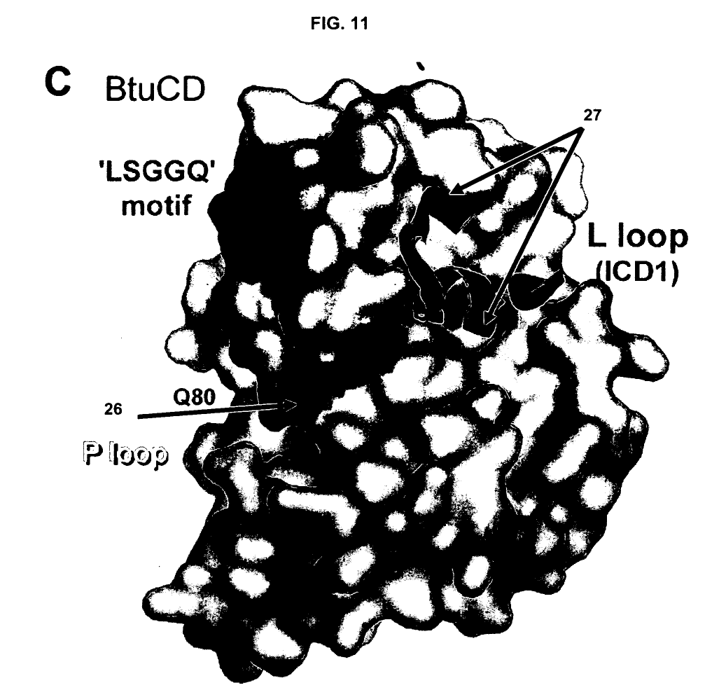

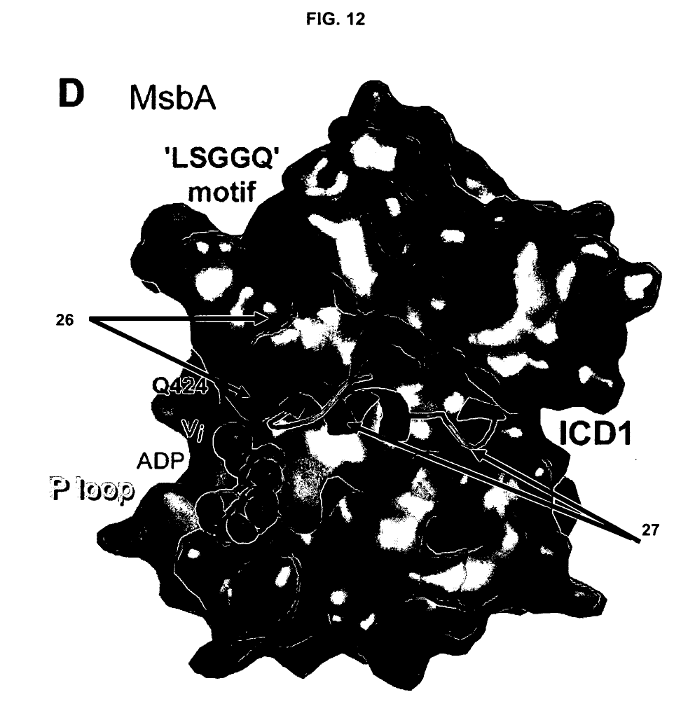

Interaction between the NBDs and the TMDs involves two conserved motifs. The Q-loop contains a conserved glutamine that coordinates Mg2+ and the proposed nucleophilic water and the short conserved ICD1 helix mediates the contacts with the proximal NBD and the TMD. In the apo structure of BtuCD, the ICD1-equivalent helix (termed the L loop) does not contact the conserved glutamine (Gln80) in the Q-loop (FIG. 11). However in the structure, the amino end of the ICD1 (residues 111-121) is in direct contact with the glutamine from the Q-loop and fits within an elongated groove on the TMD exposed NBD surface (FIG. 12). Because ICD1 is the only structural motif from the TMD to interact with the active site of the NBD, a reorientation of the ICD1 helix to interact with the Q-loop could depend on the catalytic status of the γ-phosphate.

Together with previous MsbA structures, the present structure provides a framework for interpreting functional data concerning MDR-ABC transporters. Substrate recognition by the TMD and nucleotide binding by the NBD changes the conformation of the molecule promoting the formation of the NBD dimer in an arrangement competent to hydrolyze ATP. This dimerization of the NBDs and hydrolysis of ATP is the “power stroke” of the transport cycle and drives the transport of the lipid substrate from the inner to the outer membrane leaflet through conformational changes in the TMDs. In the post hydrolysis conformation structure, only one ADP is trapped per dimer suggesting that the two NBDs act to alternately hydrolyze ATP. This would support the alternating catalytic sites model proposed for P-gp and LmrA. However, the presence of two LPS molecules on the outer leaflet side of the membrane suggests two substrate molecules may be transported per “power stroke”.

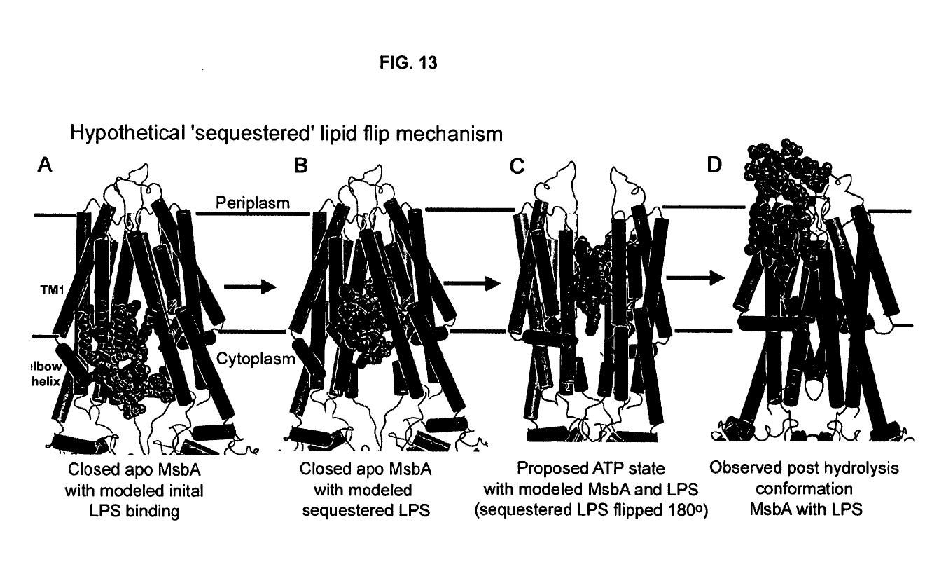

The data lead to a structurally based mechanism of LPS flipping whereby the sugar head groups are sequestered and “flipped” in the internal chamber while the hydrophobic tail of the lipid is dragged through the bilayer. The transporter has a titratable high affinity binding site for several cationic heavy metals such as 2-chloromercuri-4-nitrophenol and ethyl mercury chloride located at the interface of the elbow helix and TM1 (data not shown). Accordingly, this region, which contains a locus of conserved residues across this subfamily of MDR-ABC transporters, points to an initial high-affinity surface binding site for LPS and other cationic hydrophobic compounds similar in chemical structure to most anti-cancer chemotherapeutics. In this model, LPS initially binds near the elbow helix (FIG. 13). During the “power stroke” step, the sugar head groups are sequestered within the chamber and “flipped” to the outer membrane leaflet by the rigid body shearing of the TMDs while the hydrophobic tails of the LPS are dragged through the lipid bilayer (FIG. 13). The result is an energetically favorable “flip-flop” in the orientation of the lipid in the bilayer and the presentation of LPS sugar head groups on the periplasmic side of the membrane (FIG. 13) as observed in the structure.

The size of the chamber is large enough to accommodate the sugar groups from two Ra LPS molecules. The head group size of various LPS and related molecules effects the stimulation of MsbA ATPase activity. A larger stimulatory effect on ATP activity is observed for Kdo2-lipid A which has a shorter head group than for Ra LPS. This might be an effect of steric hindrance inside the cavity to accommodate the sugar head groups. The model also extends to other molecules with cationic/hydrophobic properties such as most chemotherapeutic drugs associated with MDR. A mechanism where export could occur more exclusively through the chamber for less hydrophobic molecules is also possible. The model described constitutes a general molecular basis for export by MDR ABC flippases and the structural characterization of a Mg.ADP.Vi post-hydrolysis conformation of MsbA providing an excellent springboard for further studies.

Drug design strategies as specifically described above with regard to residues and regions of the ligand-complexed MsbA crystal can be similarly applied to the other ABC Transporter structures, including other ABC Transporter and MDR structures, whether prokaryotic or eukaryotic, disclosed herein. One of ordinary skill in the art, using the art recognized modeling programs and drug design methods, many of which are described herein, can modify the ABC Transporter design strategy according to differences in amino acid sequence. For example, this strategy can be used to design compounds which regulate MDR in other ABC Transporters. In addition, one of skill in the art can use lead compound structures derived from one ABC Transporter, such as MsbA, and take into account differences in amino acid residues in other ABC Transporters, such as, for example, MDR1. Example 2 below bears this out.

In the present method of structure-based drug design, it is not necessary to align a candidate chemical compound (i.e., a chemical compound being analyzed in, for example, a computational screening method of the present invention) to each residue in a target site. Suitable candidate chemical compounds can align to a subset of residues described for a target site. In some configurations of the present invention, a candidate chemical compound can comprise a conformation that promotes the formation of covalent or noncovalent crosslinking between the target site and the candidate chemical compound. In certain aspects, a candidate chemical compound can bind to a surface adjacent to a target site to provide an additional site of interaction in a complex. For example, when designing an antagonist (i.e., a chemical compound that inhibits the binding of a ligand to an ABC Transporter by blocking a binding site or interface), the antagonist can be designed to bind with sufficient affinity to the binding site or to substantially prohibit a ligand (i.e., a molecule that specifically binds to the target site) from binding to a target area. It will be appreciated by one of skill in the art that it is not necessary that the complementarity between a candidate chemical compound and a target site extend over all residues specified here.

In various aspects, the design of a chemical compound possessing stereochemical complementarity can be accomplished by means of techniques that optimize, chemically or geometrically, the “fit” between a chemical compound and a target site. Such techniques are disclosed by, for example, Sheridan and Venkataraghavan, Acc. Chem Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

Some embodiments of the present invention for structure-based drug design comprise methods of identifying a chemical compound that complements the shape of an ABC Transporter or a structure that is related to an ABC Transporter. Such method is referred to herein as a “geometric approach”. In a geometric approach of the present invention, the number of internal degrees of freedom (and the corresponding local minima in the molecular conformation space) can be reduced by considering only the geometric (hard-sphere) interactions of two rigid bodies, where one body (the active site) contains “pockets” or “grooves” that form binding sites for the second body (the complementing molecule, such as a ligand).

The geometric approach is described by Kuntz et al., J. Mol. Biol., vol. 161, p. 269, 1982, which is incorporated by this reference herein in its entirety. The algorithm for chemical compound design can be implemented using a software program such as AutoDock, available from the The Scripps Research Institute (La Jolla, Calif.). One or more extant databases of crystallographic data (e.g., the Cambridge Structural Database System maintained by University Chemical Laboratory, Cambridge University, Lensfield Road, Cambridge CB2 IEW, U.K. or the Protein Data Bank maintained by Rutgers University) can then be searched for chemical compounds that approximate the shape thus defined. Chemical compounds identified by the geometric approach can be modified to satisfy criteria associated with chemical complementarity, such as hydrogen bonding, ionic interactions or Van der Waals interactions.

In some embodiments, a therapeutic composition of the present invention can comprise one or more therapeutic compounds. For example, a PSC-833 composition is provided in U.S. Pat. No. 6,239,102, incorporated herein by reference in its entirety, which can be administered in combination with a therapeutic composision of the present invention. A therapeutic composition can further comprise other compounds capable of reducing MDR. A therapeutic composition of the present invention can be used to treat disease in an animal such as, for example, a human in need of treatment by administering such composition to an animal. Non-limiting examples of animals to treat include mammals, marsupials, reptiles and birds, humans, companion animals, food animals, zoo animals and other economically relevant animals (e.g., race horses and animals valued for their coats, such as chinchillas and minks). Additional animals to treat include dogs, cats, horses, cattle, sheep, swine, chickens, turkeys. Accordingly, in some aspects, animals to treat include humans, dogs and cats.

A therapeutic composition of the present invention can also include an excipient, an adjuvant and/or carrier. Suitable excipients include compounds that the animal to be treated can tolerate. Examples are provided in U.S. Pat. No. 6,239,102. Other examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, a therapeutic composition can include a carrier. Carriers include compounds that increase the half-life of a therapeutic composition in the treated animal. Suitable carriers include, but are not limited to, polymeric controlled release vehicles, biodegradable implants, liposomes, bacteria, viruses, other cells, oils, esters, and glycols.

Acceptable protocols to administer therapeutic compositions of the present invention in an effective manner include individual dose size, number of doses, frequency of dose administration, and mode of administration. Determination of such protocols can be accomplished by those skilled in the art. Modes of administration can include, but are not limited to, subcutaneous, intradermal, intravenous, intranasal, oral, transdermal, intraocular and intramuscular routes.

Antibodies

Antibodies that specifically recognize and bind to a ABC Transporter protein, e.g. MsbA, are useful in various aspects of the present teachings. As used herein, the term “antibody” refers to polyclonal antibodies, monoclonal antibodies, and antibody fragments which are reactive against antigens. Accordingly, an antibody can be, in non-limiting example, Fab, F(ab′)2, F(ab′)2, F(ab′)3, Fc, single chain Fv (scFv), scFV-Fc, (scFv)2, dsFv, Vh, Vl, Minibody, Diabody, Triabody, or Tetrabody antibody. Methods for Engineering, producing, purifying, fragmenting, conjugating and using various types of antibodies are well known in the art, and can be found in references such as Carter (2006) Nat Rev Immunol. 6(5), 343-357; Teillaud (2005) Expert Opin Biol Ther. 5(Supp. 1) S15-27; Subramanian, ed. (2004) Antibodies: Volume 1: Production and Purification, Springer, ISBN 0306482452; Lo, ed. (2003) Antibody Engineering Methods and Protocols, Humana Press, ISBN 1588290921; Ausubel et al., ed. (2002) Short Protocols in Molecular Biology 5th Ed., Current Protocols, ISBN 0471250929; Brent et al., ed. (2003) Current Protocols in Molecular Biology, John Wiley & Sons Inc, ISBN 047150338X; Coligan (2005) Short Protocols in Immunology, John Wiley & Sons, ISBN 0471715786; Holliger, P, et al., Proc Natl Acad Sci U S A. 90: 6444-6448, 1993; Holliger, P. et al., Nature Biotechnology 23: 1126-1136, 2005; Subramanian, ed. (2001) Manufacturing of Gene Therapeutics—Methods, Processing, Regulation and Validation, Springer, ISBN 0306466805; Lo, ed. (2003) Antibody Engineering Methods and Protocols, Humana Press, ISBN 1588290921.

Also, the antibody fragments described herein can be reformatted through high-throughput methods to, for example, IgG molecules. See e.g., Carter et al. Nat Rev Immunol. 6: 343-357, 2006.

Antibodies described herein can include covalent modifications. Non-limiting examples of covalently modified antibodies include antibodies conjugated with one or more labels such as a fluorophore, a radionuclide, or a hapten (such as biotin), pegylated antibodies, antibodies conjugated with a toxin, and antibodies conjugated with a protein such as an enzyme, for example, horseradish peroxidase, alkaline phosphatase, chloramphenicol acetyltransferase, or luciferase. Antibodies can also be comprised by a non-covalent complex, for example an antibody-colloidal gold complex.

Antibody Production

Antibodies can be produced in mammalian cells such as Chinese hamster ovary cells, NSO mouse myeloma cells or hybridoma cells. Other hosts for production can also be employed (e.g., E. coli, other microorganisms, insect cells, and transgenic plants and animals). See generally Carter et al. (2006) Nat Rev Immunol. 6(5), 343-357. For example, antibody fragments can be produced at gram-per-liter titres by E. coli fermentation.

The antibody-based proteins described herein can also be produced by fusion of the genes encoding the antibodies with the genes encoding other proteins or protein fragments, such as enzymes, multimerization domains, and toxins.

Once produced, an antibody can be tested for antigen recognition by various procedures such as Western blotting, ELISA or immunoprecipitation analysis, as described in the references above. In various aspects, antigen-binding affinity (Kd ) of an antibody can range from about 10-6 M to about 10-15 M; from about 10-8 M to about 10-12 M; or from about 3.2×10-10 M to about 8×10-11 M.

Numerous properties of the antibodies described herein can be tuned to improve their clinical and/or diagnostic efficacy. These properties include, but are not limited to, immunogenicity, antigen binding specificity and affinity, effector functions and other biological activities, pharmacokinetics, molecular architecture, internalization after cell binding, and biophysical properties. For example, display libraries and structure-based design (i.e., rational design) can be used, either individually or in combination, for the optimization of antibody therapeutics. See e.g., Carter et al. (2006) Nat Rev Immunol. 6(5), 343-357, 348; Wu et al. (2005) Nature Biotech 23(9), 1137-1146. Such optimization can include, for example, minimizing adverse-immunogenicity risk; improving antigen selectivity; increasing species cross-reactivity; increasing or decreasing antigen binding affinity; increasing potency; increasing or decreasing effector functions; increasing or decreasing plasma half-life; increasing or decreasing internalization efficiency; increase chemical, proteolytic, and thermodynamic stability; and improve solubility and folding kinetics. As a specific example, phage-display libraries can be used to select antibody fragments optimized for robust expression, high stability, and solubility. As another specific example, phage-display libraries can be used for affinity maturation of antibodies and increased in vitro biological potency. See e.g., Carter et al. (2006) Nat Rev Immunol. 6(5), 343-357, 350. Such increases can increase efficacy as well as reduce dosage or frequency of administration.

The terminal half-life of antibodies in plasma can be tuned over a wide range to fit clinical goals. The antibody half-life can be engineered to be between several minutes to several weeks. It can also be desirable to increase the terminal half-life of an antibody to improve efficacy, to reduce the dose or frequency of administration, or to improve localization to the target. Alternatively, it can be advantageous to do the converse—that is, to decrease the terminal half-life of an antibody—to reduce whole body exposure or to improve the target-to-non-target binding ratios.

Monoclonal antibody fragments can be engineered to tailor pharmacokinetics and allow selection of optimized versions for various applications, such as imaging or therapeutics. See e.g., Wu et al. (2005) Nature Biotech 23(9) 1137-1146. For example, scFVs (around 25 kDa) are diabodies (around 55 kDa) below the threshold for first-pass renal clearance and can show terminal half-lives on the order of, for example, several hours. Larger fragments such as minibodies or small immunoproteins (e.g., scFVs fused to single constant domains of IgG, 80 kDa) show intermediate clearance rate and reach higher tissue uptake levels. Still larger fragments include scFVs fused to intact Fc domains (scFVs-Fcs, 110-120 kDa) have similar pharmokinetics to intact monoclonal antibodies because of the Fc region, including the neonatal Fc receptor bidning site.

Decreasing antibody terminal half-life can, for example, allow tumor imaging by positron emission tomography. For example, the terminal half-life of IgG can be increased or decreased by tailoring the interaction between IgG and its salvage receptor, FcRn. See e.g., Carter et al. (2006) Nat Rev Immunol. 6(5), 343-357, 353. The terminal half-life of antibody fragments, usually shorter than non-fragments, can be extended by binding to longer-lived molecules, such as IgG and serum albumin, or conjugation to molecules such as polyethylene glycol (i.e., PEGylation).

Conjugation of antibodies to a variety of agents, including drugs, toxins, and radionuclides, is well known in the art. See generally, Wu et al. (2005) Nat Biotechnol. 23(9), 1137-1146; McCarron et al. (2005) Mol Interv 5(6), 368-380; Niemeyer (2004) Bioconjugation Protocols, Strategies and Methods, Humana Press, ISBN 1588290980; Hermanson (1996) Bioconjugate Techniques, Academic Press, ISBN 0123423368.

Antibody Uses

Among the various uses for antibodies described herein are, for example, function inhibitor, expression inhibitor, detector agent, diagnostic agent, purification agent, therapeutic agent, and imaging agent.

Antibodies of the present teachings can be used as inhibitors of adenosine triphosphate binding cassette transporter function and expression. Inhibitors of ABC transporters include inhibitors targeting various domains of an ABC protein, such as a drug binding site, a nucleotide binding site. Standard methods using antibodies can be used to detect and quantify ABC transporter expression, including but not limited to: radioimmunoassays, receptor assays, enzyme immunoassays, cytochemical bioassays, ligand assays, immunoradiometric assays, fluoroimmunoassays, and enzyme-linked immunosorbent assays.

The antibodies described herein can be used to detect the presence and quantity of the substance against which they were raised. For example, the antibodies of the invention can be used to detect ABC transporter protein in a biological sample. Various protocols well known in the art can be utilized for these purposes, including but not limited to, a Western blot analysis or immunofluorescence staining. For example, antibodies described herein can be used in an immunoassay to monitor the level of an ABC transporter protein in a cell or tissue sample).

Antibodies described herein can be used as therapeutic agents, either alone or conjugated to another active agent. Therapeutic use of antibodies is known in the art. See e.g., Carter (2006) Nat Rev Immunol. 6(5), 343-357; Subramanian, ed. (2004) Antibodies vol. 2 Novel Technologies and Therapeutic Use, Springer, ISBN 0306483157. Antibodies can also be modified for delivery of, for example, a toxin, radioisotope, cytokine or other active conjugate. Bispecific antibodies can also be designed to bind both to a target antigen and to a conjugate or effector cell.

Antibodies of the invention can also be used in a variety of imaging and/or localization applications.

The methods discussed above are well known and will be understood by those skilled in the art to require a reasonable amount of experimentation to optimize the interaction between antibodies and antigens and the detection of the antigens by the antibodies. See e.g., Wild (2005) The Immunoassay Handbook, 3d ed., Elsevier Science, ISBN 0080445268; Coligan (2005) Short Protocols in Immunology, John Wiley & Sons, ISBN 0471715786; Brent et al., ed. (2003) Current Protocols in Molecular Biology, John Wiley & Sons Inc, ISBN 047150338X; Ausubel et al., ed. (2002) Short Protocols in Molecular Biology 5th Ed., Current Protocols, ISBN 0471250929.

Radioimmaging and Radioimmunotherapy

Radionuclides can be coupled to the antibodies described herein, thus facilitating a variety of imaging and therapeutic protocols. See e.g., Wu et al. (2005) Nature Biotech 23(9), 1137-1146. As an imaging example, redioimmunoscintigraphy using gamma cameras or single photon emmission tomography requires coupling of gamma emitting isotopes (e.g., 99mTc, 123I, 111In) to an antibody. Positron emission tomogrpahy (PET) relies on attachment of positron emitters (e.g., 18F, 64Cu, 68Ga, 86Y, 124I) to antibodies. Targeted delivery of beta emitters (e.g., 131I, 90Y, 177Lu, 67Cu) or alpha-emitting radionucleotides (e.g., 213Bi, 211At) through conjugation to an antibody is a touchstone for effective radioimmunotherapy procedures. Radioimmunoimaging can be used in conjunction with radioimmunotherapy as a means for evaluating targeting and dosimetry. Generally, antibody fragments can be used for immunoimaging applications.

Antibodies can be coupled to radionuclides for radioimmunotherapy applications. Generally, dose delivered to the target is balanced against exposure of normal organs and tissues to radiation. Both alpha and beta emitters with a variety of energy transfer properties, half-lives and emission rates can be used for radioimmunotherapy. See e.g., Milenic et al. (2004) Nat Rev Drug Discov 3, 488-499. Examples of toxic radionuclides that can be coupled to the antibody include 131I, 90Y, and 177Lu.

Coupling radionucleotides and proteins is well known in the art and can be accomplished, for example, through conjugation to exisiting or genetically introduced cysteine residues in the antibody. See e.g., Wu et al. (2005) Nature Biotech 23(9), 1137-1146.; McCarron et al. (2005) Mol Interv 5(6), 368-380; Niemeyer (2004) Bioconjugation Protocols, Strategies and Methods, Humana Press, ISBN 1588290980; Hermanson (1996) Bioconjugate Techniques, Academic Press, ISBN 0123423368. Other examples of antibody modifications include labeling of hexahistidine-tagged recombinant proteins and covalent modification of monoclonal antibody binding sites for ligand binding. A further coupling example is incorporation of enyzmatically labile linkers between radiometal and antibody (e.g., Wu, A.M., et al., Nature Biotechnology 23: 1137-1146, 2005.

Antibodies of the present teachings can also be used in pretargeting radioimmunotherapy. See e.g., Sharkey et al. (2005) Clin Cancer Res 11, 7109s-7121s. In addition, the antibodies can be used antibody-directed enzyme prodrug therapy (ADEPT). See e.g., Wu et al. (2005) Nature Biotech 23(9), 1137-1146.

Humanization of Antibodies

Antibodies of the present teachings include chimeric, humanized, and fully human antibodies. Such agents can be produced through protein engineering (e.g., complementarity-determining region grafting), through library technologies (e.g., phage, yeast, or ribsome display), or by MAb generation in transgenic mice.

Complete human antibodies can be derived from, for example, phage-display technology or transgenic mice that express human immunoglobulin genes. See e.g. Carter et al. (2006) Nat Rev Immunol. 6(5), 343-357, 346-347. Phage-display selection technology can also be utilized to, for example, optimize binding affinity; select for specific binding properties, such as species cross-reactivity; or obtain large (e.g., over 1,000) collections of specific antibodies to identify combinatorial properties or very high potency. Antibody fragments produced via phage display can be reformatted through high-throughput methods into various other forms of antibodies, such as IgG, as known in the art. Other examples for generation of human antibodies include ribosome-, mRNA- and yeast-display libraries, as well as human hybridomas from patients and antibody-cDNA cloning from single lymphocytes selected on antigen.

Antibody Administration

The antibodies described herein can be used therapeutically either as exogenous materials or as endogenous materials. As used herein, exogenous agents are those produced or manufactured outside of the body and administered to the body, while endogenous agents are those produced or manufactured inside the body by some type of device (biologic or other).

Administration of antibodies by a variety of methods is well known in the arts. Antibody delivery can be by intravenous infusion, usually entailing multiple doses. Local, controlled release methods for antibody delivery are also known in the art. See e.g., See e.g., Raza et al. (2005) Expert Opin Biol Ther. 5(4), 477-494; Stayton et al. (2005) Orthod Craniofacial Res 8, 219-225; Grainger (2004 Expert Opin Biol Ther. 4(7), 1029-44; Varde and Pack (2004) Expert Opin Biol Ther. 4(1), 35-51; Whittlesey and Shea (2004) Exp Neurol. 190(1),1-16.

Antibodies of the present teachings for use in therapy can be administered in an amount of, for example, from about 1 μg per injection to about 1 mg per injection, from about 10 μg per injection to about 100 μg per injection, or from about 20 μg per injection to about 50 μg per injection.

Adverse reactions to antibody administration in a subject can be attenuated in a variety of ways known in the art. For example, infusion reactions (e.g., fever, chills, headaches, vomiting, and diarrhoea) can be attenuated by humanization, attentuating effector functions (e.g., antibody-dependent cell-mediated cytotoxicity and complement dependent cytotoxicity), premedication, and by incremental increase in the rate of infusion of antibody formulation. See e.g. Carter et al. (2006) Nat Rev Immunol. 6(5), 343-357. As another example, Fc-mediated reactions, such as acute and severe influenza-like syndrome, can be largely overcome by attenuating the interaction between the Fc region of the antibody and the receptors for the antibody (e.g., IgG receptors; FcγRs) expressed by the patient. In general, increasing the potency of the antibody or extending its half-life in plasma can allow the dose or frequency of administration to be reduced, with the associated benefits of improved quality of life and/or convenience for the patient, and/or reduced cost of the drug.

Aptamers

In various configurations of the present teachings, aptamers can be used as binding agents with specificity for an ABC transport. Aptamers are nucleic acid binding species with affinities and specificities for protein targets similar to those of monoclonal antibodies. Generation, selection, and delivery of aptamers is discussed in, for example, Lee et al., Curr Opin Chem Biol. (Apr. 15, 2006).

“Smart” Polymeric Carriers

The biomolecular therapeutic agents described herein can be delivered to intracellular targets via so-called “smart” polymeric carriers. See e.g., Stayton et al. (2005) Orthod Craniofacial Res 8, 219-225; Wu et al. (2005) Nature Biotech (2005) 23(9), 1137-1146. Generally, carriers of this type utilize polymers that are hydrophilic and stealth-like at physiological pH, but become hydrophobic and membrane-destabilizing after uptake into the endosomal compartment (i.e., acidic stimuli from endosomal pH gradient) where they enhance the release of the cargo molecule into the cytoplasm. The design of the smart polymeric carrier can incorporate pH-sensing functionalities, hydrophobic membrane-destabilizing groups, versatile conjugation and/or complexation elements to allow the drug incorporation, and an optional cell targeting component. Potential therapeutic macromolecular cargo includes, but is not limited to, peptides, proteins, antibodies, polynucleotides, plasmid DNA (pDNA), aptamers, antisense oligodeoxynucleotides (ASODN), silencing RNA, and ribozymes. As an example, smart polymeric carriers can enhance the cytoplasmic delivery of antibody-targeted conjugates that are internalized through receptor mediated endocytosis. As another example, smart polymeric carriers can enhance cytoplasmic delivery of protein therapeutics.

Polymeric carriers include, for example, the family of poly(alkylacrylic acid) polymers, specific examples including poly(methylacrylic acid), poly(ethylacrylic acid) (PEAA), poly(propylacrylic acid) (PPAA), and poly(butylacrylic acid) (PBM), where the alkyl group progressively increased by one methylene group.

Such polymeric carriers can be designed to provide a range of pH profiles and membrane-destabilizing activities, allowing their molecular properties to be matched to specific drugs and loading ranges. For example, the pH profile can be controlled by the choice of the alkylacrylic acid monomer and by ratio of the carboxylate-containing alkylacrylic acid monomer to alkylacrylate monomer. Similarly, the membrane destabilizing activity can be controlled by the lengths of the alkyl segment on the alkylacrylic acid monomer and the alkylacrylate monomer.

Smart polymeric carriers with potent pH-responsive, membrane destabilizing activity can be designed to be below the renal excretion size limit. For example, poly(EM-co-BA-co-PDSA) and poly(PAA-co-BA-co-PDSA) polymers exhibit high hemolytic/membrane destabilizing activity at the low molecular weights of 9 and 12 kDa, respectively.

Various linker chemistries are available to provide degradable conjugation sites for proteins, nucleic acids, and/or targeting moieties. For example, pyridyl disulfide acrylate (PDSA) monomer allow efficient conjugation reactions through disulfide linkages that can be reduced in the cytoplasm after endosomal translocation of the therapeutics.

TABLE 3
Data processing, phasing and refinement statistics

| | | | |
|---|---|---|---|
| MsbA | MsbA CMNP | MsbA-2′Br-ATP | MsbA-Vᵢ |

Data processing

| Source (wavelength, Å) | ALS 8.3.1 | SSRL 9-1 | ALS 8.3.1 | ALS 8.3.1 |
|---|---|---|---|---|
| | | (λ = 1.006 Å) | (λ = 0.9198 Å) | (λ = 2.2608 Å) |
| Space group | C2 | | | |
| Unit cell, Å | a = 271.4 b = 122.0 c = 177.9 β = 121.7° | | | |
| Resolution, Å | 4.2 | 5.5 | 5 | 5.7 |
| Redundancy | 3.3 | 3.1 | 2.8 | 2 |
| R_{sym}^{a}, % | 6.7 | 7.5 | 6.8 | 8.9 |
| Completeness, % | 85 | 98.5 | 85 | 81 |

Phasing statistics

| | |
|---|---|
| Phasing power | 1.68 |
| Density correlation | 35% (dimer 1) |
| coefficient between | 40% (dimer 2) |
| NCS related dimers | |
| Overall figure of merit | 0.35 (SAD) |

Model Building and Refinement

| | |
|---|---|
| R_{cryst}^{b}, % | 28 |
| R_{free}^{c}, % | 33 |
| Rmsd^{d} bond lengths, Å | 0.02 |
| Rmsd^{d} bond angles, deg | 1.7 |
| <B>, Å² | 90 |

^{a}R_{sym} = Σ|I − <I>|Σ<I>, where I is the measured intensity of each reflection, and <I> is the intensity averaged from symmetry equivalents.
^{b}R_{cryst} = Σ|Fₒ − F_{c}|/Σ|F_{c}|, where Fₒ and F_{c} are observed and calculated structure factors, respectively.
^{c}R_{free} was calculated from a test set (10%) omitted from the refinement.
^{d}Rmsd, root mean square deviation.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### Crystalization of Transporter-LPS-Nucleotide Complex

A. Purification and Crystallization

MsbA from Salmonella typhimurium was isolated by PCR from purified genomic material. MsbA homologs were cloned into the pET19b expression vector (Novagen, Madison, Wis.), which contains a 23-residue fusion leader containing an N-terminal deca-histidine tag to aid in purification and expressed in E. coli BL21 (DE3) (Novagen, Madison, Wis.) in a 100 liter batch fermentor at 37° C. using 2 mM IPTG (Anatrace, Maumie, Ohio) as the inducer. MsbA was extracted from E. coil by agitation in the presence of 1-1.2% (w/v) undecyl-β-D-maltoside (β-UDM) at 4° C. Extracted MsbA was purified in the presence of 20 mM Tris-HCL (pH 8.0), 20 mM NaCl, and 0.05% β-UDM in the presence of 10% glycerol. Additional rounds of chromatography were required to increase protein enrichment and purity. Purified MsbA was assayed for purity by Commassie blue staining and mass spectrometry, and then concentrated to 15-20 mg/ml protein with a final concentration 0.05% β-UDM.

B. Crystallization and Crystallographic Data Collection

An approach was adopted of screening several detergents and bacterial orthologs using methods that were successful in solving previous structures of MsbA. As a result of this work, crystals were obtained of MsbA with ADP.Vi from several species in different detergents and crystallization conditions. The crystals of MsbA from S. typhimurium (ST-MsbA) in complex with ADP.Vi and Ra LPS grew in space group C2 with unit cell dimensions of a=271.4 Å, b=122.0 Å, c=121.7 Å, α=90°, β=121.7°, γ=90°. Detergent solubilized protein was preincubated with 10 mM Ra LPS purified from S. typhimurium. In addition, 15 mM ATP, 20 mM MgCl₂, and 1 mM sodium ortho-vanadate (boiled for five minutes prior to use) were added prior to crystallization to promote the transition state conformation. Large-scale crystallization efforts screening different temperatures, detergents, precipitants, salts, and additives were explored. ST-MsbA crystals were grown using the sitting drop method at 4°, by combining protein with precipitant at a ratio of 2:1. The precipitant solution consisted of 100 mM Tris-HCL (pH 7.5-8.0), 100 mM NaCl, 325 NaCH₃COO and 19% PEG 400. Crystals appeared after two weeks and continued to grow to a full size in approximately one month. To verify the identity of the sample, the crystals were washed, dissolved, and verified by SDS-PAGE gel. Mass spectrometry analysis confirmed the predicted molecular mass of the protein and the Ra LPS.

C. Data Collection

Data collection parameters and statistics are listed in Table 3. Initial crystal screening and native data were collect to a resolution of 4.5 Å at the Stanford Synchrotron Radiation Laboratory (SSRL) beamline 11-1 and Advance Photon Source (APS) beamline BM-14 (data not shown). ST MsbA-CMNP crystals diffracted to 5.5 Å and a two wavelength (λ=1.006 Å and λ=1.009 Å) MAD data set was collected at the SSRL 11-3. The 4.2 Å native data, the ST MsbA—2′Br ATP data and the vanadium-edge data were all collected at Advance Light Source in Berkeley (ALS) beamline 8.3.1. All data collected were processed with HKL2000 (S1). The coordinates, Table 1 herein, are deposited with the Protein Databank under Accession No. 1Z2R.

D. X-ray Structure Determination

Protein phases were determined by single anomalous scattering at the mercury LIII edge using a mercury derivative. To produce the derivative, MsbA was preincubated and buffer exchanged with 10 mM 2-chloromercuri-4-nitrophenol (CMNP) and crystallized as previously described. Data from a single wavelength was used because of the severe radiation damage to the crystals during data collection. Initial electron density maps clearly revealed that the asymmetric unit contained two ST-MsbA transporters (four monomers) corresponding to a solvent/detergent content of ˜70%. Electron density correlations on experimentally derived maps indicated that there were some differences between monomers as well as between the dimers of the transporter. These structural differences were accommodated in the averaging masks and density modification procedures. Iterative 2-fold non-crystallographic symmetry averaging between dimers, solvent flattening/flipping, and phase extension using computer package PHASES (S2) yielded electron density maps of high quality that enabled us to definitively identify the TMD, NBD, LPS, ADP and vanadate positions. A chemical model was built using the program CHAIN (S3) and MOLOC (S4). The LPS molecule from the structure of FhuA and E. coli K-12 LPS, was used as a starting model for Ra LPS (S5). Crystallographic refinement using XPLOR (v.3.851) (S6) initially, followed by CNS (v1.1) (S7), resulted in a model of R and R_{free} of 28% and 33%, respectively to 4.2 Å in resolution.

E. Graphics

The figures were produced with the program Pymol (S7).

### Example 2

### Crystalization of Transporter-PSC833-Nucleotide Complex

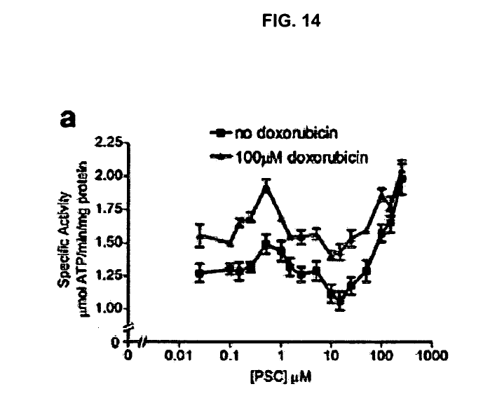

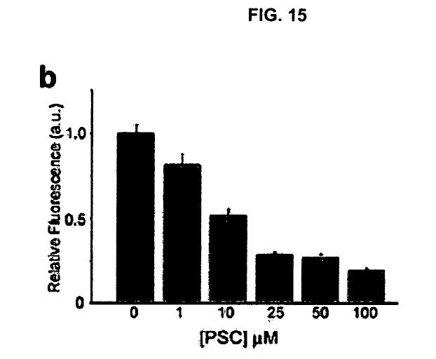

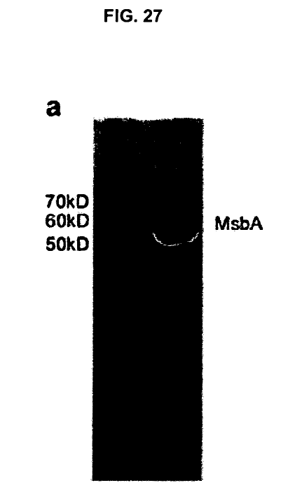

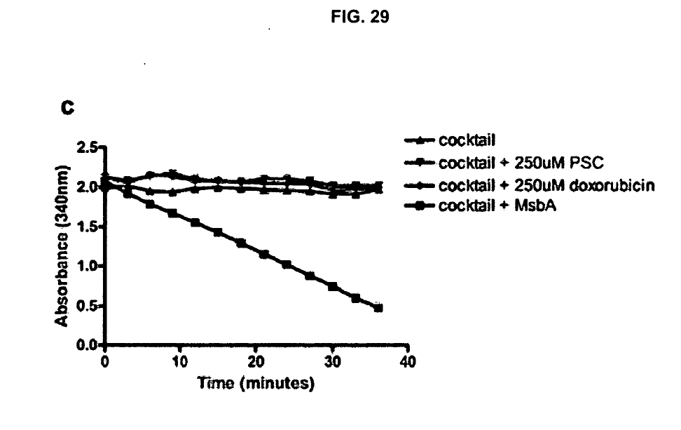

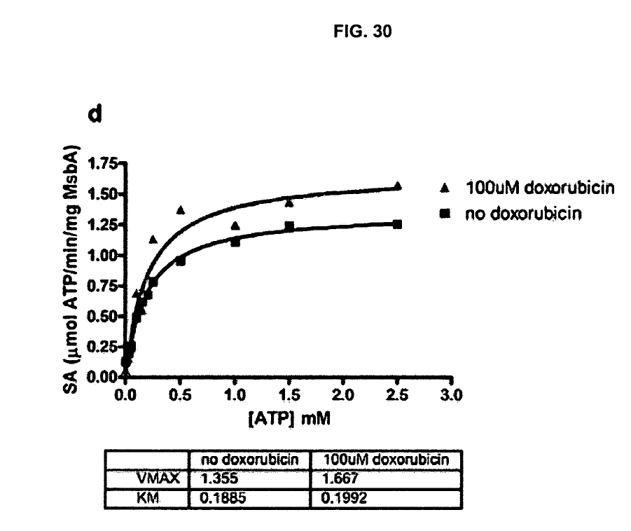

This example illustrates the X-ray structure of MsbA, in complex with cyclosporin analogue PSC-833 and ADP.vanadate, and reveals insights into the biology of drug efflux and the mechanism of transport inhibition. PSC-833 (PSC), a cyclosporine D analogue, is a potent inhibitor of P-gp mediated drug accumulation in human and murine MDR cell lines. PSC modulates the ATPase activity of purified MsbA. ATPase activity of detergent solubilized MsbA (Reyes, C. L. & Chang, G., Acta Crystallograph. Sect. F. Struct. Biol. Cryst. Commun. 61: 655-658, 2005) was measured using a linked enzyme assay (Vogel, G. & Steinhart, R., Biochemistry 15, 208-216 (1976); Urbatsch, I. L., et al., J. Biol. Chem. 270: 19383-19390, 1995) and shows a basal activity of 1.25 μmol ATP/min/mg of protein. In this study, we observe that MsbA ATPase activity is modulated at concentrations less than 50 μM (FIG. 14). At concentrations of 50-250 μM, PSC stimulates ATPase up to 55% over basal activity (FIG. 14). A similar ATPase profile was observed in different orthologs of MsbA and reconfirmed with a separate ATPase assay directly measuring inorganic phosphate release (data not shown). Doxorubicin stimulates ATPase activity in a dose dependent manner with maximum effect at 100 μM (see FIGS. 27-30). PSC also modulates doxorubicin stimulated ATPase activity of MsbA (FIG. 14). Controls are shown in FIGS. 27-30. In addition, PSC alters the retention of doxorubicin over time in an E. coli strain overexpressing MsbA (FIG. 15).

Crystals of MsbA with PSC were grown as described (Reyes, C. L. & Chang, G., Acta Crystallograph. Sect. F. Struct. Biol. Cryst. Commun. 61: 655-658, 2005) using detergent solubilized protein incubated with ATP, Mg²⁺, sodium ortho-vanadate, Ra LPS, and PSC. The structure was determined by molecular replacement using the electron density from the MsbA.ADP.vanadate.Ra LPS crystal form for initial phases (Reyes, C. L. & Chang, G., Science 308: 1028-1031, 2005). Electron density maps were improved by non-crystallographic symmetry averaging to a resolution of 4.5 Å. Although the diffraction resolution is modest, the key features are well resolved in the electron density including the 1.2 kD PSC molecule, large aromatic sidechain positions, and the conformational rearrangements of the transmembrane helices and loops. Interestingly, Ra LPS was present throughout the crystallization process, however no density for LPS was observed in the PSC crystal form suggesting a loss of affinity due to conformational changes in the TMDs. A chemical model with good geometry was built with Rcryst of 28% and Rfree of 39% (see Table 4).

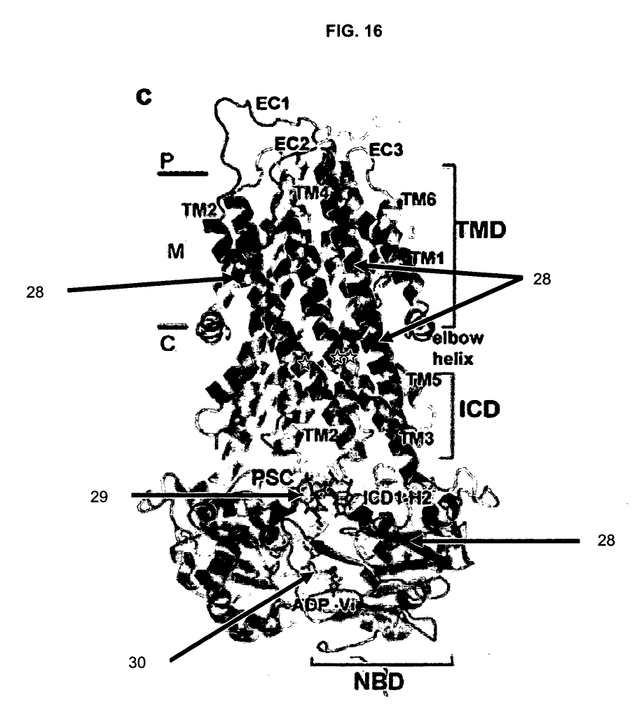

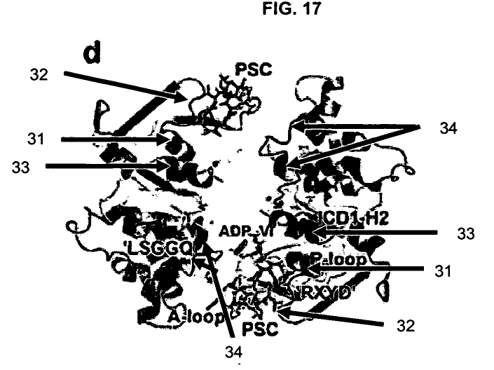

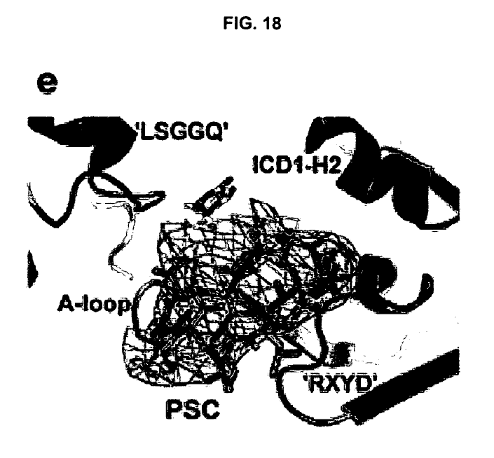

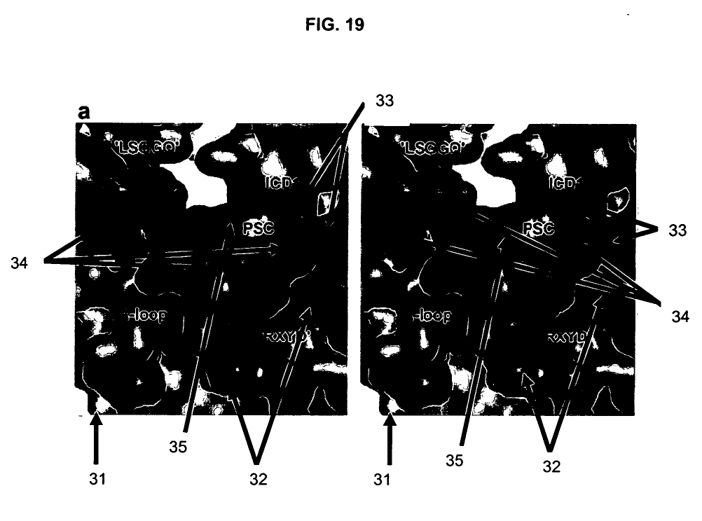

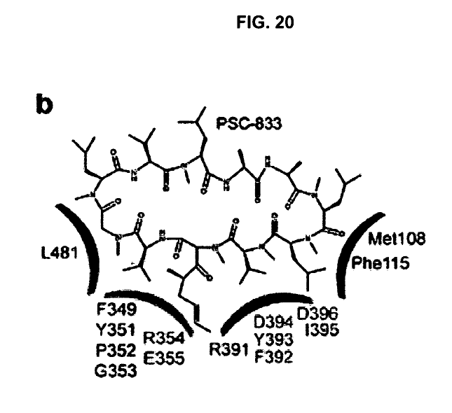

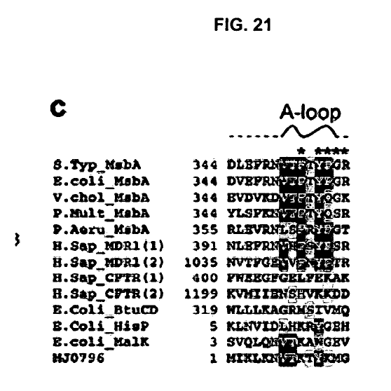

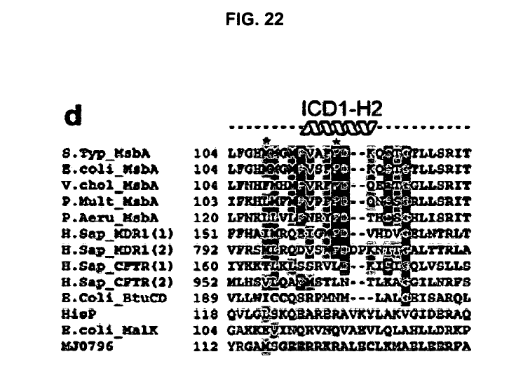

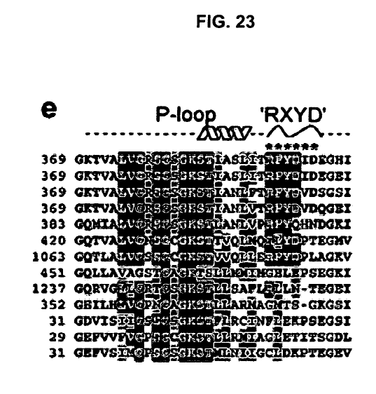

Analysis of the X-ray structure of a complex comprising MsbA and PSC-833 indicates that MsbA is arranged as a homodimer composed of two interacting transmembrane domains (TMDs) and two nucleotide binding domains (NBDs) (FIGS. 16 and 17). Electron density for PSC was observed in a pocket formed between the ATP binding site and the second helix of intracellular domain 1 (ICD1-H2; residues 111-121) in each monomer (FIG. 18). ICD1-H2 is formed between the cytoplasmic ends of TM2 and TM3. ICD1-H2 makes extensive contacts with conserved sequence motifs in the NBDs. PSC buries ˜40% (˜440 Å) (Tai, H. L., Curr. Opin. Mol. Ther. 2: 459-467, 2000) of its surface area by making van der Waals interactions with a sequence motif following the P-loop (‘RXYD’ loop), the ICD1, the ‘LSGGQ’ signature motif, and the ATP coordinating A-loop (FIGS. 18 and 19). A more detailed analysis shows that side chains within 6 Å of PSC include: loop 4 residues Arg391, Phe392, Tyr393, Asp394, Ile395 and Asp396; ICD1 residues Met108 and Phe115; ‘LSGGQ’ motif residue Leu481; and Aloop residues Phe349, Thr350, Tyr351, Pro352, Gly353, Arg354 and Glu355; (FIG. 20). The ICD1, A-loop, and ‘RXYD’ loop that form the binding pocket of PSC are conserved in both MsbA and P-gp but vary considerably in other ABC transporters (FIGS. 21-23).

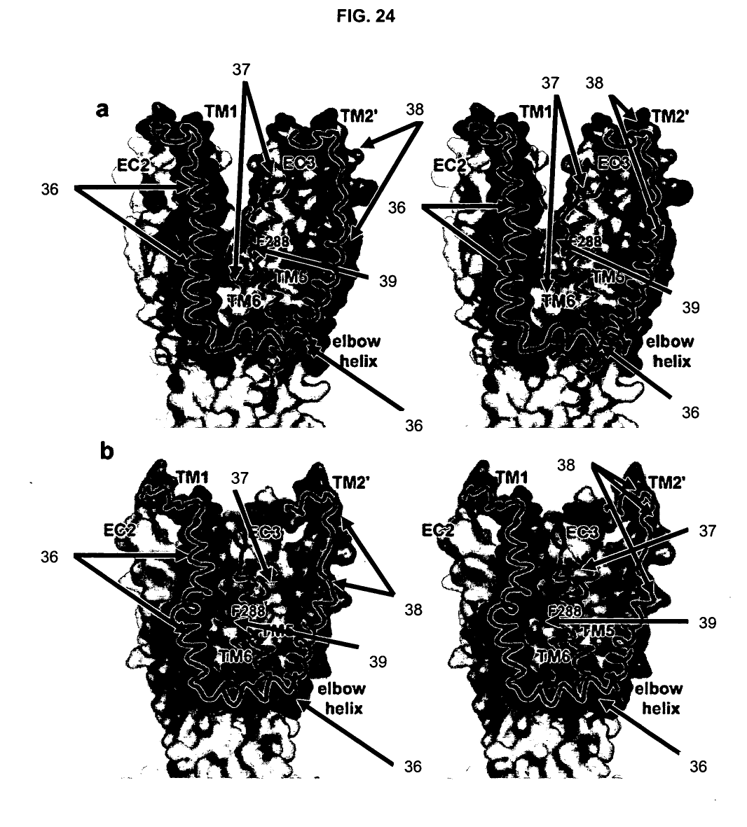

Binding of PSC near the ATP binding sites causes significant structural changes in the TMDs. The binding of PSC causes a structural ‘kink’ in both the TM2 and TM3 helices at the TMD/ICD interface (FIG. 17). This ‘kink’ pushes the second extracellular loop (EC2) away from the periplasmic end of TM1 and induces a significant bend in TM1 towards TM6. In the MsbA.ADP.Vi.LPS structure, TM1 and TM6 form a ‘V’ shaped opening with accessibility to the periplasmic side of the membrane (FIG. 24). In the PSC structure, this opening is closed as TM1 packs against TM6 (FIG. 24). TM5 from one monomer also interacts more extensively with TM5 from the opposite monomer, disrupting accessibility to an internal chamber.

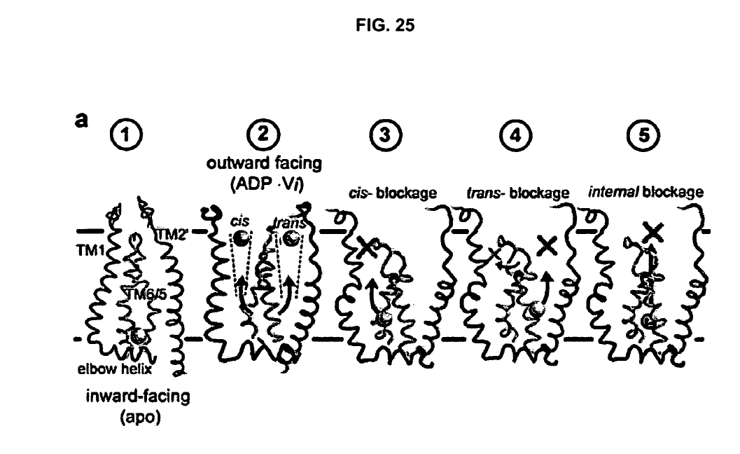

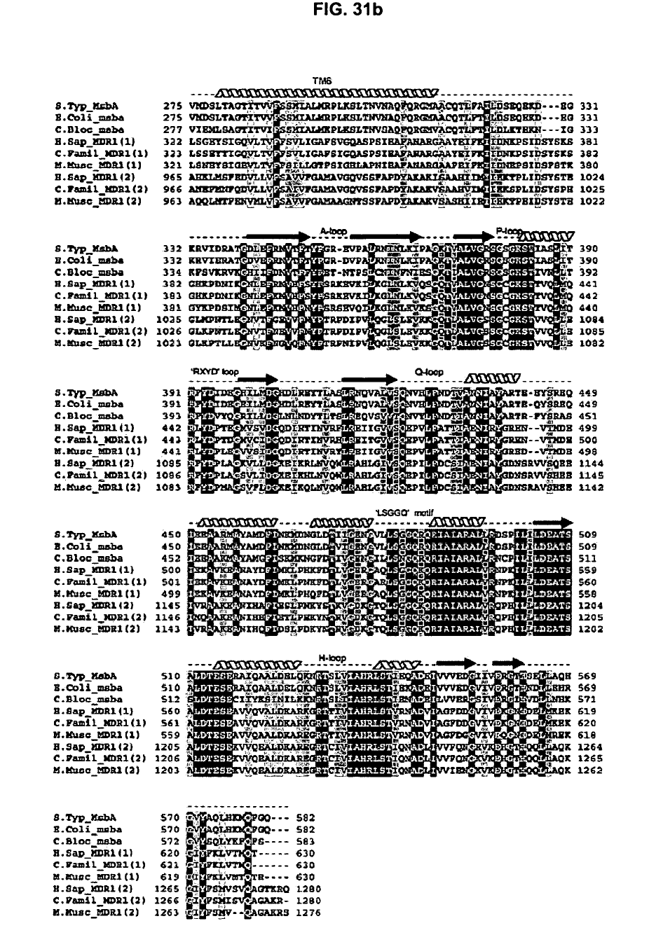

Rearrangements in the TMD helices provide insight into the mechanism of substrate transport and inhibition. Structural comparisons of the TMDs observed in MsbA.PSC.ADP.vanadate to MsbA.ADP.vanadate.Ra LPS and apo MsbA (Chang, G., J. Mol. Biol. 330: 419-430, 2003), reveals two grooves termed the cis- and trans-grooves, that are formed by helix-helix interactions formed by TM5-TM6 and bracketed on either side by TM1cis and TM2trans (cis-same monomer, trans-opposite monomer) (FIG. 25). Presumably, PSC binding disrupts transport by closing the periplasmic ends of these grooves. In the MsbA.Ra LPS structure, lipid binds to the extracellular loops centered on TM6. The absence of electron density for LPS in the PSC data is likely a consequence of the conformational changes observed in these grooves. Genetic analysis of P-gp mutants that affect drug resistence and homology modeling have mapped key residues to both the cis- and trans-grooves (Shilling, R. A. et al., Trends Pharmacol. Sci. 27: 195-203, 2006). One such residue, Phe335 (human P-gp), maps to residue Phe288 in S. typhimurium MsbA (FIG. 31) and is implicated as a key residue in transport regulation (Venter, H., et al., Nature 426, 866-870, 2003) (FIG. 24). Interestingly, this residue is located at the packing interface between TM1 and TM6, and may control the passage of substrate from the inner leaflet side of the cis-groove. We propose that mutations in this region will disrupt the close helix-helix packing interface required to mediate the transport in either groove. In addition, the outward-facing opening of the internal chamber is closed in this structure and suggests blockage of direct transport through an internal pathway. Transport through an internal pathway has been previously speculated for ATP-independent ethidium bromide efflux by LmrA, a prokaryotic ABC transporter with functional homology to MsbA and P-gp (Venter, H., et al., Nature 426: 866-870, 2003).

The conformational changes caused by PSC reveal insight into the mechanism coupling ATP hydrolysis to substrate transport. A Glyl85Val mutation in P-gp, significantly decreases transport of chemotherapeutic drugs and increases binding of photoreactive analogs of these compounds, while increasing basal ATPase activity (Safa, A. R. et al., Proc. Natl. Acad. Sci. USA 87: 7225-7229, 1990). One interpretation is that mutations at or near this site might decouple drug stimulated ATP hydrolysis from drug transport. In MsbA, this residue maps to the strictly conserved Gly141 which is located at the site of the ‘kink’ in TM3 and is likely involved in the transmission of signal between the NBD and TMD (FIG. 16). A second strictly conserved glycine (Gly94 in MsbA corresponding with Gly141 in P-gp) is also located at the ‘kink’ in TM2 and together with Gly141 may allow structural flexibility required for transmitting signal at the TMD/ICD interface. Based on this structure, PSC may disrupt this signaling mechanism by decoupling hydrolysis from transport.

The PSC binding site resolved in this x-ray structure is well situated to disrupt the catalytic cycle of transport by interacting with the nucleotide binding pocket and ICD1. We propose that the ATPase activity profile observed is a consequence of multiple binding events of PSC that alternately affect the integrity of the interactions between the nucleotide, ICD1s and conserved NBD motifs. The doxorubicin retention data suggest that transport inhibition is independent of ATPase modulation. Therefore, our data suggest that transport may be disrupted at low concentrations of PSC by decoupling ATP hydrolysis in the NBDs from transport pathways in the TMDs. Additional inhibitory effects likely occur at high concentrations of PSC through competitive binding to substrate binding sites in the TMDs. In this conformation with ADP and vanadate, we do not observe interpretable density for PSC in the TMDs. Perhaps this is a consequence of a lower affinity for substrate in the initial binding site due to conformational changes associated with the post-hydrolysis state captured in this structure.

The structural and functional analysis of MsbA with PSC provides a molecular structural basis for the modulation of ATPase activity and transport inhibition. PSC binds in a pocket near the the catalytic machinery uncoupling ATP hydrolysis from substrate transport. Other cyclic peptide compounds, such as dendroamides and patellamides, have been reported to be inhibitors of P-gp (Ogino, J., et al., J. Nat. Prod. 59: 581-586, 1996) and MsbA and may also have similar binding modes. In conclusion, this pocket represents a unique target in the development of MDR inhibitors as well as potent antibiotics that specifically target MsbA in Gram-negative pathogens such as Yersinia pestis and Pseudomonas aeruginosa.

Purification and crystallization. Purification and crystallization experiments are similar to those described in Reyes and Chang (Acta Cystallograph. Sect. F. Struct. Biol. Cryst. Commun. 61: 655-658, 2005). Purified MsbA was concentrated to 15 mg/ml in YM100 Centricon filters (Millipore) and assayed for purity by Commassie blue staining (FIG. 27). Detergent solubilized protein from S. typhimurium in β-UDM was preincubated with 10 mM LPS (purchased from Sigma Chemical Co., St. Louis, Mo.); purified from S. typhimurium), 500 μM PSC (provided by Novartis), 15 mM ATP, 2.5 mM MgCl2, and 1 mM sodium ortho-vanadate. Crystal drops were grown using the sitting drop method at 4° C., by combining protein with precipitant at a ratio of 2:1. The precipitant solution contained 100 mM Tris-HCL (pH 7.5-8.0), 100 mM NaCl, 325 mM NaCH₃COO, and 18-24% PEG 400.

Data collection and structure determination. Data were collected to a resolution of 4.5 Å at Advance Light Source (ALS) beamline 8.2.2 (Table 4).

TABLE 4
Data processing and refinement statistics
MsbA-PSC-ADP-Vanadate
Data collection and processing

| Source | ALS 8.2.2 |
|---|---|
| Space group | C2 |
| Unit cell, Å | a = 268.2 b = 121.4 |
| | c = 176.2 β = 121.2° |
| Resolution, Å | 4.5 |
| Redundancy | 4.1 |
| Rsym^{a}, % | 6.3 |
| Completeness, % | 84 |
| I/σ(I) | 4.6 |

Model Building and Refinement

| | |
|---|---|
| Rcryst^{b}, % | 28 |
| Rfree^{c}, % | 39 |
| Rmsd^{d} bond lengths, Å | 0.01 |
| Rmsd^{d} bond angles, deg | 2.2 |
| <B>, Å² | 100 |

^{a}Rsym = Σ|I − <I>|Σ<I>, where I is the measured intensity of each reflection, and <I> is the intensity averaged from symmetry equivalents.
^{b}Rcryst = Σ|Fo − Fc|/Σ|Fc|, where Fo and Fc are observed and calculated structure factors, respectively.
^{c}Rfree was calculated from a test set (7%) omitted from the refinement.
^{d}Rmsd, root mean square deviation.

All data collected were processed with HKL2000 (Otwinowski, Z. & Minor, W., Methods in Enzymology 276: 307-326, 1997). The structure was determined by molecular replacement using electron density from the previously determined structure of MsbA.ADP.vanadate.Ra LPS (Reyes, C. L. & Chang, G. Science 308: 1028-1031, 2005). Subsequent electron density maps were improved by using iterative four fold non-crystallographic symmetry averaging. The PSC binding site was clearly resolved in our averaged maps. An atomic model of PSC based on the known chemical structure was built into electron density near the ATP binding pocket in both NBDs. The position and quality of this electron density was confirmed by a polyalanine omit map of PSC and the whole model. No electron density for LPS was observed. Crystallographic refinement using CNS (v1.1) (Brunger, A. T. et al. Acta Crystallogr. D. Biol. Crystallogr. 54: 905-921, 1998) resulted in a model of Rcryst of 28% and Rfree of 39%. Figures were prepared with PyMol (DeLano, W. L. The PyMOL Molecular Graphics System. 2002; http://pymol.sourceforge.net/).

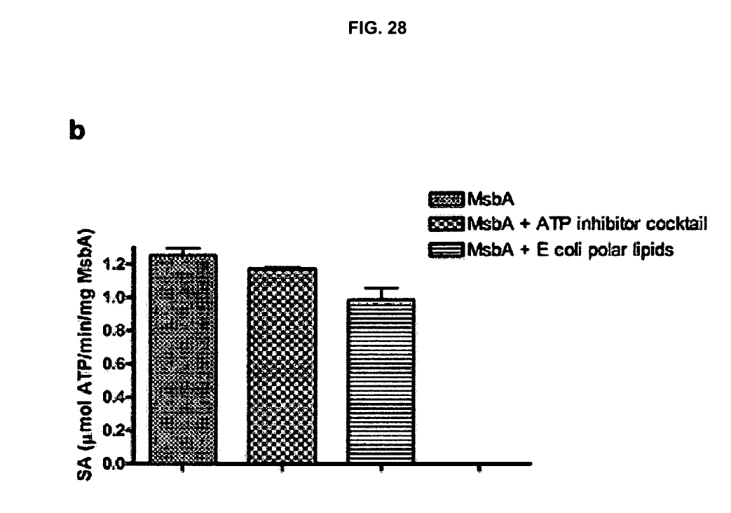

ATPase Activity. ATPase activity was measured using an ATP-regenerating system described by Vogel and Steinhart, and modified by Urbatsch et al. Briefly, 1 μg of MsbA was added to 100 μl of Linked Enzyme (LE) buffer at 37° C. containing 10 mM ATP, 12 mM MgCl₂, 6 mM phosphoenolpyruvate (PEP), 1 mM NADH, 10 units of lactate dehydrogenase (LDH), 10 units of pyruvate kinase (PK), and 50 mM Tris-HCl, pH 7.5. ATP hydrolysis was measured as the decrease in absorbance of NADH at 340 nm using a DXT880 multiplate spectrofluorimeter (Beckman-Coulter). Activity of MsbA in the presence of either E. coli polar lipids (10 μg/ml) or a non-ABC, ATPase inhibitor cocktail (1 mM EGTA, 1 mM NaN3, 0.2 mM ouabain) was measured (FIGS. 28-30). Each data point is the average of 5 independent measurements with standard error bars shown.

Doxorubicin Retention. A pET 15b vector containing the msbA gene from Salmonella typhimurium LT2 was transformed into an E. coli strain deficient in the multiple-drug efflux pump AcrAB (ECΔacrAB). Single colony cultures were grown to optical density (OD600 nm) of 0.6 in the presence of 1 mM IPTG to induce overexpression of MsbA, harvested and washed three times with 100 mM potassium phosphate buffer (pH 7.5) containing 5 mM MgSO4 and concentrated to an OD600 of 20. Cellular accumulation of doxorubicin was accomplished by incubation of cells with 11.5 μM doxorubicin and 40 μM carbonyl cyanide m-chlorophenylhydrazone (CCCP) while shaking for 1 hour at 37° C. The cells were then washed three times and incubated for 15 minutes at 37° C. with 40 μM CCCP and 0, 1, 10, 25, 50 or 100 μM PSC. Efflux was initiated by addition of 25 mM glucose and fluorescence in the cell suspensions was monitored at 16-second time intervals with a DXT880 multiplate spectrofluorimeter (Beckman-Coulter) with excitation at 485 nm and emission at 595 nm for 900 seconds. PSC exhibits no fluorescence at these wavelengths. Cellular doxorubicin retention was measured as a change of fluoresence at time=400 sec versus time=0 sec. Control cells without addition of glucose exhibited no change of fluorescence. Wild type E. coli cells showed doxorubicin retention less than 5 times compared to untreated ECΔacrAB cells and exhibited a less than 10% increase in retention with 50 μM PSC. Results are the average of 3 experiments, with 3-8 measurements per experiment. Statistical significance was assigned with the two-way analysis of variance (ANOVA). A P<0.05 is considered significant.

Sequence Alignments. Amino acid sequence alignments in FIGS. 21-23 and FIG. 31 were produced using the program CLUSTALW. The amino acid sequence alignments in FIGS. 21-23 include MsbA from Salmonella typhimurium (SEQ ID NO: 17), Escherichia coli (SEQ ID NO: 18), Vibrio cholerae (SEQ ID NO: 19), Pasteurella multocida (SEQ ID NO: 20) and Pseudomonas aeruginosa (SEQ ID NO: 21); both halves of MDR1 (SEQ ID NO: 22) and CFTR (SEQ ID NO: 23) from Homo sapiens; BtuC/D (SEQ ID NOs: 24 and 25 respectively), HisP (SEQ ID NO: 26) and MalK (SEQ ID NO: 27) from Escherichia coli; and MJ0796 from Methanococcus jannaschii (SEQ ID NO: 28). We also aligned amino acid sequences of MsbA from Salmonella typhimurium (SEQ ID NO: 17), Escherichia coli (SEQ ID NO: 18) and Candidatus blochmannia (SEQ ID NO: 29) and both halves of MDR1 and CFTR (SEQ ID NOs: 22 and 23 respectively) from Homo sapiens, Mus. musculus (SEQ ID NO: 30) and Canis familiaris (SEQ ID NO: 31), shown in FIG. 31. GenBank Accession Numbers are provided for these sequences in Table 5 below:

TABLE 5
GenBank Accession Nos. for Species MsbA/MDR1 Sequences

| | GenBank | SEQ |
|---|---|---|
| Species | Accession No. | ID NO: |
| Salmonella typhimurium MsbA | NP_459959 | 17 |
| Escherichia coli MsbA | CAA77839 | 18 |
| Vibrio cholerae MsbA | AAF95026 | 19 |
| Pasteurella multocida MsbA | AAK02945 | 20 |
| Pseudomonas aeruginosa PAO1 MsbA | NP_253684 | 21 |
| Homo sapiens MDR1 | NP_000918 | 22 |
| Homo sapiens CFTR | P13569 | 23 |
| Escherichia coli BtuC | P06609 | 24 |
| Escherichia coli BtuD | AAN80565 | 25 |
| Escherichia coli HisP | NP_754734 | 26 |
| Escherichia coli MalK | AAB59057 | 27 |
| Methanocaldococcus jannaschii, MJ0796 | Q58206 | 28 |
| Candidates Blochmannia floridanus MsbA | CAD83445 | 29 |
| Canis familiaris MDR1 | NP_001003215 | 30 |
| Mus musculus MDR1 | NP_035205 | 31 |

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Specifically incorporated herein by reference in their entirety are: Reyes C L, Chang G. Structure of the ABC transporter MsbA in complex with ADP.vanadate and lipopolysaccharide. Science. May 13, 2005;308(5724): 1028-31; Reyes C L, Chang G. Lipopolysaccharide stabilizes the crystal packing of the ABC transporter MsbA. Acta Crystallograph Sect F Struct Biol Cryst Commun. Jul. 1, 2005;61 (Pt 7):655-8. Epub Jun. 15, 2005; and Reyes C L, Ward A, Yu J, Chang G. The structures of MsbA: Insight into ABC transporter-mediated multidrug efflux. FEBS Lett. Feb. 13, 2006;580(4):1042-8. Epub Dec. 1, 2005.

Publications incorporated herein by reference in their entirety include:

1. R. C. Moellering Jr., Clin. Infect. Dis. 16, 135 (1998).

2. M. Ouellette, D. Legare, B. Papadopoulou, J. Mol. Microbiol. Biotechnol. 3, 201 (2001).

3. M. Dean and R. Allikmets, J. Bioenerg. Biomembr. 33, 475 (2001).

4. H. W. van Veen, C. F. Higgins, W. N. Konings, Res. Microbiol. 152, 365 (2001).

5. V. Ling, Cancer 69, 2603 (1992).

6. Y. Raviv, H. Pollard, E. P. Bruggermann, I. Pastan, M. M. Gottesman, J. Biol. Chem. 265, 3975 (1990).

7. M. Karow, C. Georgopoulos, Mol. Microbiol. 7, 69 (1993).

8. A. Polissi, C. Georgopoulos, Mol. Microbiol. 20, 1221 (1996).

9. Z. Zhou, K. A. White, A. Polissi, C. Georgopoulos, C. R. Raetz, J. Biol. Chem. 273, 12466 (1998).

10. G. Reuter, T Janvilisri, H. Venter, S. Shahi, L. Balakrishnan, H. W. van Veen, J. Biol. Chem. 278, 35193 (2003).

11. W. T. Doerrler, H. S. Gibbons, C. R. Raetz, J. Biol. Chem. 279, 45102 (2004).

12. W. T. Doerrler, C. R. Raetz, J. Biol. Chem. 277, 36697 (2002).

13. R. Medzhitov, C. A. Janeway, Cell 91, 295 (1997).

14. J. A. Hoffmann, F. C. Kafatos, C. A. Janeway, R. A. Ezekowitz, Science 284, 1313 (1999).

15. C. R. Raetz, C. Whitfield, Annu. Rev. Biochem.71, 635 (2002).

16. G. Chang and C. B. Roth, Science 293, 1793 (2001).

17. K. P. Locher, A. T. Lee and D. C. Rees, Science 296, 1091 (2002).

18. G. Chang, J. Mol. Biol. 330, 419 (2003).

19. M. F. Rosenberg, R. Callaghan, S. Modok, C. F. Higgins, R.C. Ford, J. Biol. Chem. E-pub (2004).

20. A. Ferreira-Pereira, S. Marco, A. Decottignies, J. Nader, A. Goffeau, J. L. Rigaud, J. Biol. Chem. 278, 11995 (2003).

21. J. Y. Lee, I. L. Urbatsch, A. E. Senior, S. Wilkens, J. Biol. Chem. 277, 40125 (2002).

22. M. Chami et al., J. Mol. Biol. 315, 1075 (2002).

23. M. F. Rosenberg et al., EMBO J. 20, 5615 (2001).

24. C. F. Higgins, K. J. Linton, Nat. Struct. Mol. Biol. 10, 918 (2004).

25. Materials and Methods, Supporting Online Material.

26. M. Gruen, C. Becker, A. Beste, J. Reinstein, A. J. Scheidig, R. S. Goody, Protein Sci. 8, 2524 (1999).

27. C. A. Smith, I. Rayment, Biochemistry 35, 5404 (1996).

28. J. Dong, G. Yang, H. S. Mchaourab. Submitted to Science November 2004.

29. A. H. Buchaklian, A. L. Funk, C. S. Klug, Biochemistry 43, 8600 (2004).

30. G. F. Ecker et al., Mol. Pharmacol. 66, 1169 (2004).

31. T. W. Loo, D. M. Clarke, J. Biol. Chem. 27632. P. C. Smith et al., Mol. Cell. 10, 139 (2002).

33. K. P. Hopfner et al., Cell 101, 789 (2000).

34. A. E. Senior, D. C. Gadsby, Semin. Cancer Biol. 8,143 (1997).

35. H. W. van Veen, A. Margolles, M. Muller, C. F. Higgins, W. N. Konings, EMBO J. 19, 2503 (2000).

S1. Z. Otwinowski and W. Minor, Methods in Enzymology 276, (1997).

S2. W. Furey and S. Swaminathan, Methods in Enzymology 277 (1997).

S3. J. Sacks, Mol. Graph. 6 (1988).

S4. MOLOC. Gerber Molecular Design. Amden, Switzerland.

S5. A. D. Ferguson et al., Structure Fold Des. 8 (2000).

S6. A. T. Brunger, X-PLOR, v. 3.1. Yale University Press, New Haven, Conn. (1992).

S7. A. T. Brunger et al., 54 (1998).

S8. W. L. DeLano, World Wide Web http://www.pymol.org. (2002)

What is claimed is:

1. A method for designing a drug which interferes with an activity of an Adenosine Triphosphate Binding Cassette Transporter, the method comprising:
(a) providing on a digital computer a three-dimensional structure of a Transporter Ligand Complex comprising the Adenosine Triphosphate Binding Cassette Transporter and at least one ligand of the Transporter; and
(b) using software comprised by the digital computer to design a chemical compound which is predicted to bind to the Adenosine Triphosphate Binding Cassette Transporter.

2. A method according to claim 1, further comprising:
(c) synthesizing the chemical compound; and
(d) evaluating the chemical compound for an ability to interfere with an activity of the Adenosine Triphosphate Binding Cassette Transporter.

3. A method according to claim 1, wherein the chemical compound is designed by computational interaction with reference to a three dimensional site of the structure of the Transporter-Ligand Complex, wherein the three dimensional site is selected from the group consisting of TM1: 37-44, TM2: 57-78, TM6: 277-291, EC1: 49-68, EC2: 160-168, EC3: 270-278, ICD1: 92-142, elbow helix: 10-22, TM1: 26-37, TM3: 143-153, TM4: 180-187, TM5: 251-256, TM6: 291-303, and any combination thereof.

4. A method according to claim 1, wherein the chemical compound is designed by computational interaction with reference to a three dimensional site of the structure of the Transporter-Ligand Complex, wherein the three dimensional site is selected from the group consisting of LSGGQ motif (SEQ ID NO: 32), A-loop, RXYD loop (SEQ ID NO: 33), intracellular domain 1 (ICD1) and a combination thereof.

5. A method according to claim 4, wherein the LSGGQ motif comprises L481.

6. A method according to claim 4, wherein the A-loop comprises at least one amino acid selected from the group consisting of F349, Y351, P352, G353, R354 and E355.

7. A method according to claim 4, wherein the RXYD loop comprises at least one amino acid selected from the group consisting of R391, F392, Y393, b394, 1395 and D396.

8. A method according to claim 4 wherein the ICD1 comprises at least one amino acid selected from the group consisting of M108 and F115.

9. A method for generating a model of a three dimensional structure of a Transporter-Ligand Complex, the method comprising:
(a) providing an amino acid sequence of a known Transporter-Ligand Complex and an amino acid sequence of a target Transporter-Ligand Complex;
(b) identifying structurally conserved regions shared between the known Transporter-Ligand Complex amino acid sequence and the target Transporter-Ligand Complex amino acid sequence; and
(c) assigning atomic coordinates from the conserved regions to the target Transporter-Ligand Complex.

10. A method in accordance with claim 9, wherein the known Transporter-Ligand Complex has a three dimensional structure described by atomic coordinates that substantially conform to atomic coordinates set forth in Table 1 or Table 2.

11. A method for determining a three dimensional structure of a target Transporter-Ligand Complex structure comprising:
(a) providing an amino acid sequence of a target structure, wherein the three dimensional structure of the target structure is not known;
(b) predicting the pattern of folding of the amino acid sequence in a three dimensional conformation using a fold recognition algorithm; and
(c) comparing the pattern of folding of the target structure amino acid sequence with the three dimensional structure of a known Transporter-Ligand Complex.

12. A method in accordance with claim 11, wherein the known Transporter-Ligand Complex comprises a three dimensional structure described by atomic coordinates that substantially conform to atomic coordinates set forth in Table 1 or Table 2.

13. A method for treating a microbial infection, the method comprising administering to a patient in need thereof a therapeutically effective amount of a composition comprising an inhibitor of an Adenosine Triphosphate Binding Cassette Transporter and an antibiotic.

14. A method in accordance with claim 13, wherein the inhibitor is an inhibitor selected by performing a structure based drug design using a three-dimensional structure determined for a crystal of the Adenosine Triphosphate Binding Cassette.

15. A method in accordance with claim 13, wherein the inhibitor is an inhibitor selected by
a) performing a structure based drug design using a three-dimensional structure determined for a crystal of the Adenosine Triphosphate Binding Cassette to identify a candidate inhibitor;
b) contacting the candidate inhibitor with the Adenosine Triphosphate Binding Cassette Transporter; and
c) detecting inhibition of at least one activity of the Transporter.

16. A method in accordance with claim 15, wherein the Adenosine Triphosphate Binding Cassette Transporter is a prokaryotic Adenosine Triphosphate Binding Cassette Transporter.

17. A method in accordance with claim 16, wherein the prokaryotic Adenosine Triphosphate Binding Cassette Transporter is a bacterial Adenosine Triphosphate Binding Cassette Transporter.

18. A method in accordance with claim 17, wherein the bacterial Adenosine Triphosphate Binding Cassette Transporter is a gram negative bacterial Adenosine Triphosphate Binding Cassette Transporter.

19. A method in accordance with claim 18, wherein the gram negative bacterial Adenosine Triphosphate Binding Cassette Transporter is a Salmonella Adenosine Triphosphate Binding Cassette Transporter.

20. A method in accordance with claim 19, wherein the Salmonella Adenosine Triphosphate Binding Cassette Transporter is a Salmonella typhimurium msbA Adenosine Triphosphate Binding Cassette Transporter.

21. A method in accordance with claim 13, wherein the inhibitor is an antibody directed against the Adenosine Triphosphate Binding Cassette Transporter.

22. A method in accordance with claim 21, wherein the inhibitor is an antibody directed against the Salmonella typhimurium msbA Adenosine Triphosphate Binding Cassette Transporter.

23. A method in accordance with claim 22, wherein the antibody is a monoclonal antibody.

24. A method in accordance with claim 21, wherein the antibody is directed against at least one extracellular loop domain.

25. A method in accordance with claim 24, wherein the at least one extracellular loop domain comprises a peptide sequence selected from the group consisting of

| | |
|---|---|
| kpllddgfgktdrsvllwmp, | (SEQ ID NO: 1) |
| mfyyswqls | (SEQ ID NO: 2) |
| and | |
| asfpsvmds. | (SEQ ID NO: 3) |

26. A method in accordance with claim 21, wherein the antibody is directed against at least one domain of the Adenosine Triphosphate Binding Cassette Transporter which contacts a substrate.

27. A method in accordance with claim 26, wherein the at least one domain of the Adenosine Triphosphate Binding Cassette Transporter which contacts a substrate comprises a peptide sequence selected from the group consisting of naasdtfm (SEQ ID NO: 4), gktdrsvllwmplvviglmilr (SEQ ID NO: 5) and dsltagtitvvfssm (SEQ ID NO: 6).

28. A method in accordance with claim 21, wherein the antibody is directed against at least one intercellular domain of the Adenosine Triphosphate Binding Cassette Transporter.

29. A method in accordance with claim 28, wherein the at least one intercellular domain comprises a peptide sequence
(SEQ ID NO: 7)
vsgkvvmtmrrrlfghmmgmpvaffdkqstgtllsritydseqvassssg
a.

30. A method in accordance with claim 21, wherein the antibody is directed against at least one drug-binding domain of the Adenosine Triphosphate Binding Cassette Transporter.

31. A method in accordance with claim 30, wherein the at least one drug-binding domain of the Adenosine Triphosphate Binding Cassette Transporter comprises a peptide sequence selected from the group consisting of wqtfrrlwptiap (SEQ ID NO: 8); glivagialiln (SEQ ID NO: 9); litvvregasi (SEQ ID NO: 10); iairvvsk (SEQ ID NO: 11); sdpiiq (SEQ ID NO: 12) and mialmrplksltn (SEQ ID NO: 13).

32. A method in accordance with claim 21, wherein the antibody is directed against at least one extracellular domain of the Adenosine Triphosphate Binding Cassette Transporter.

33. A method in accordance with claim 32, wherein the at least one extracellular domain of the Adenosine Triphosphate Binding Cassette Transporter comprises a peptide sequence selected from the group consisting of ddgfgktdrsvl (SEQ ID NO: 14), wql (SEQ ID NO: 15), and psvmdslt (SEQ ID NO: 16).

34. A method in accordance with claim 15, wherein the inhibition of at least one activity of the Transporter comprises an inhibition of activity selected from the group consisting of ATP hydrolysis, transmembrane transport of a cationic hydrophobic compound, binding of the cationic hydrophobic compound to the Transporter and release of the cationic hydrophobic compound from the Transporter.

35. A method in accordance with claim 34, wherein the cationic hydrophobic compound is a lipopolysaccharide.

36. A method in accordance with claim 35, wherein the lipopolysaccharide is a rough type lipopolysaccharide selected from the group consisting of an Ra rough type lipopolysaccharide, an Rb rough type lipopolysaccharide, an Rc rough type lipopolysaccharide, an Rd rough type lipopolysaccharide and an Re rough type lipopolysaccharide.

37. A method in accordance with claim 17, wherein the bacterial Adenosine Triphosphate Binding Cassette Transporter is a gram positive bacterial Adenosine Triphosphate Binding Cassette Transporter.

38. A method for treating a microbial infection, the method comprising administering to a patient in need thereof a therapeutically effective amount of a composition comprising an anti-microbial agent covalently attached to an antibody directed against an Adenosine Triphosphate Binding Cassette Transporter.

39. A method in accordance with claim 38, wherein the anti-microbial agent is an anti-microbial peptide.

40. A method in accordance with claim 39, wherein the anti-microbial peptide is selected from the group consisting of an insect anti-microbial peptide and a vertebrate anti-microbial peptide.

41. A method in accordance with claim 40, wherein the insect anti-microbial peptide is a cecropin.

42. A method in accordance with claim 41, wherein the cecropin is a cecropin-melittin hybrid peptide.

43. A method in accordance with claim 41, wherein the vertebrate anti-microbial peptide is a mammalian anti-microbial peptide.

44. A method in accordance with claim 39, wherein the anti-microbial peptide is selected from the group consisting of a myeloid antimicrobial peptide, an alpha-defensin, a beta-defensin, a protegrin, a porcine cecropin P1, a Bac5, a Bac7, a PR-39 and a prophenin.

45. A method in accordance with claim 38, wherein the antibody directed against an Adenosine Triphosphate Binding Cassette Transporter is an antibody directed against a bacterial Adenosine Triphosphate Binding Cassette Transporter.

46. A method in accordance with claim 45, wherein the prokaryotic Adenosine Triphosphate Binding Cassette Transporter is a bacterial Adenosine Triphosphate Binding Cassette Transporter.

47. A method in accordance with claim 46, wherein the bacterial Adenosine Triphosphate Binding Cassette Transporter is a gram negative bacterial Adenosine Triphosphate Binding Cassette Transporter.

48. A method in accordance with claim 47, wherein the gram negative bacterial Adenosine Triphosphate Binding Cassette Transporter is a Salmonella Adenosine Triphosphate Binding Cassette Transporter.

49. A method in accordance with claim 48, wherein the Salmonella Adenosine Triphosphate Binding Cassette Transporter is a Salmonella typhimurium msbA Adenosine Triphosphate Binding Cassette Transporter.

50. A method in accordance with claim 38, wherein the antibody is a monoclonal antibody.

51. A method for treating a cancer, the method comprising administering to a patient in need thereof a therapeutically effective amount of a composition comprising an inhibitor of an Adenosine Triphosphate Binding Cassette Transporter and an anticancer chemotherapeutic.

52. A method in accordance with claim 51, wherein the inhibitor is an inhibitor selected by performing a structure based drug design using a three-dimensional structure determined for a crystal of the Adenosine Triphosphate Binding Cassette.

53. A method in accordance with claim 51, wherein the inhibitor is an inhibitor selected by
a) performing a structure based drug design using a three-dimensional structure determined for a crystal of the Adenosine Triphosphate Binding Cassette to identify a candidate inhibitor;
b) contacting the candidate inhibitor with the Adenosine Triphosphate Binding Cassette Transporter; and
c) detecting inhibition of at least one activity of the Transporter.

54. A method in accordance with claim 51, wherein the Adenosine Triphosphate Binding Cassette Transporter is a eukaryotic Adenosine Triphosphate Binding Cassette Transporter.

55. A method in accordance with claim 54, wherein the eukaryotic Adenosine Triphosphate Binding Cassette Transporter is a mammalian Adenosine Triphosphate Binding Cassette Transporter.

56. A method in accordance with claim 55, wherein the mammalian Adenosine Triphosphate Binding Cassette Transporter is a human Adenosine Triphosphate Binding Cassette Transporter.

57. A method in accordance with claim 56, wherein the human Adenosine Triphosphate Binding Cassette Transporter is a P-glycoprotein Adenosine Triphosphate Binding Cassette Transporter.

58. A method in accordance with claim 57, wherein the P-glycoprotein Adenosine Triphosphate Binding Cassette Transporter is an MDR1 Adenosine Triphosphate Binding Cassette Transporter.

59. A method in accordance with claim 51, wherein the inhibitor is an antibody directed against the Adenosine Triphosphate Binding Cassette Transporter.

60. A method in accordance with claim 51, wherein the inhibitor is an antibody directed against the MDR1 Adenosine Triphosphate Binding Cassette Transporter.

61. A method in accordance with claim 59, wherein the antibody is a monoclonal antibody.

62. A method in accordance with claim 59, wherein the antibody is directed against at least one extracellular loop domain comprised by the Adenosine Triphosphate Binding Cassette Transporter.

63. A method in accordance with claim 59, wherein the antibody is directed against at least one domain of the Adenosine Triphosphate Binding Cassette Transporter which contacts a substrate.

64. A method in accordance with claim 59, wherein the antibody is directed against at least one intercellular domain of the Adenosine Triphosphate Binding Cassette Transporter.

65. A method in accordance with claim 59, wherein the antibody is directed against at least one drug-binding domain of the Adenosine Triphosphate Binding Cassette Transporter.

66. A method in accordance with claim 59, wherein the antibody is directed against at least one extracellular domain of the Adenosine Triphosphate Binding Cassette Transporter.

67. A method in accordance with claim 53, wherein the inhibition of at least one activity of the Transporter comprises an inhibition of an activity selected from the group consisting of ATP hydrolysis, transmembrane transport of a cationic hydrophobic compound, binding of the cationic hydrophobic compound to the Transporter and release of the cationic hydrophobic compound from the Transporter.

68. A method in accordance with claim 67, wherein the cationic hydrophobic compound is the anticancer chemotherapeutic.

69. A method in accordance with claim 68, wherein the anticancer chemotherapeutic is selected from the group consisting of colchicine, doxorubicin, adriamycin, vinblastine, digoxin, saquinivir and paclitaxel.

70. A method for treating a microbial infection, the method comprising administering to a patient in need thereof a therapeutically effective amount of an inhibitor of an Adenosine Triphosphate Binding Cassette Transporter covalently attached to an anti-microbial agent.

71. A method in accordance with claim 70, wherein the inhibitor is an inhibitor selected by performing a structure based drug design using a three-dimensional structure determined for a crystal of the Adenosine Triphosphate Binding Cassette.

72. A method in accordance with claim 70, wherein the inhibitor is an inhibitor selected by
a) performing a structure based drug design using a three-dimensional structure determined for a crystal of the Adenosine Triphosphate Binding Cassette to identify a candidate inhibitor;
b) contacting the candidate inhibitor with the Adenosine Triphosphate Binding Cassette Transporter; and
c) detecting inhibition of at least one activity of the Transporter.

73. A method in accordance with claim 70, wherein the Adenosine Triphosphate Binding Cassette Transporter is a bacterial Adenosine Triphosphate Binding Cassette Transporter.

74. A composition in accordance with claim 73, wherein the bacterial Adenosine Triphosphate Binding Cassette Transporter is a gram negative bacterial Adenosine Triphosphate Binding Cassette Transporter.

75. A method in accordance with claim 74, wherein the bacterial Adenosine Triphosphate Binding Cassette Transporter is a Salmonella typhimurium msbA Adenosine Triphosphate Binding Cassette Transporter.

76. A method in accordance with claim 72, wherein the inhibition of at least one activity of the Transporter comprises an inhibition of activity selected from the group consisting of ATP hydrolysis, transmembrane transport of a cationic hydrophobic compound, binding of the cationic hydrophobic compound to the Transporter and release of the cationic hydrophobic compound from the Transporter.

77. A method in accordance with claim 76, wherein the cationic hydrophobic compound is a lipopolysaccharide.

78. A method in accordance with claim 77, wherein the lipopolysaccharide is a rough type lipopolysaccharide selected from the group consisting of an Ra rough type lipopolysaccharide, an Rb rough type lipopolysaccharide, an Rc rough type lipopolysaccharide, an Rd rough type lipopolysaccharide and an Re rough type lipopolysaccharide.

79. A method in accordance with claim 73, wherein the bacterial Adenosine Triphosphate Binding Cassette Transporter is a gram positive bacterial Adenosine Triphosphate Binding Cassette Transporter.

80. A method in accordance with claim 70, wherein the anti-microbial agent is an anti-microbial peptide.

81. A method in accordance with claim 80, wherein the anti-microbial peptide is selected from the group consisting of an insect anti-microbial peptide and a vertebrate anti-microbial peptide.

82. A method in accordance with claim 81, wherein the insect anti-microbial peptide is a cecropin.

83. A method in accordance with claim 82, wherein the cecropin is a cecropin-melittin hybrid peptide.

84. A method in accordance with claim 81, wherein the vertebrate anti-microbial peptide is a mammalian anti-microbial peptide.

85. A method in accordance with claim 80, wherein the anti-microbial peptide is selected from the group consisting of a myeloid antimicrobial peptide, an alpha-defensin, a beta-defensin, a protegrin, a porcine cecropin P1, a Bac5, a Bac7, a PR-39 and a prophenin.

86. A Transporter-Ligand Complex in a crystalline form.

87. A complex according to claim 86, wherein the Transporter-Ligand Complex comprises MsbA, LPS and a nucleotide.

88. A complex according to claim 87, wherein the complex is arranged in a space group C2 so as to form a unit cell of dimensions a=271.4 Å, b=122.0 Å, c=121.7 Å, α=90°, β=121.7°, γ=90°.

89. A complex in accordance with claim 88, wherein the MsbA is Salmonella typhimurium MsbA.

90. A complex according to claim 86, wherein the Transporter-Ligand Complex comprises MsbA and a cyclosporin D analogue.

91. A complex according to claim 90, wherein the cyclosporin D analogue is PSC-833.

92. A complex according to claim 90, further comprising a nucleotide.

93. A complex according to claim 92, wherein the nucleotide is ADP.

94. A complex according to claim 91, wherein the complex is arranged in a space group C2 so as to form a unit cell of dimensions a=268.2 Å, b=121.4 Å, c=176.2 Å, β=121.2°.

95. A Transporter-Ligand Complex crystal, wherein said crystal is sufficiently pure to determine atomic coordinates of the ligand-complexed MsbA by X-ray diffraction to a resolution of about 4.5 Å.

96. A Transporter-Ligand Complex crystal according to claim 95, wherein said crystal is sufficiently pure to determine atomic coordinates of the ligand-complexed MsbA by X-ray diffraction to a resolution of about 4.2 Å.

97. A composition for treating a microbial infection, the composition comprising:
an inhibitor of an Adenosine Triphosphate Binding Cassette Transporter; and
an antibiotic.

98. A composition in accordance with claim 97, wherein the inhibitor is an inhibitor selected by performing a structure based drug design using a three-dimensional structure determined for a crystal of the Adenosine Triphosphate Binding Cassette.

98. A composition in accordance with claim 97, wherein the inhibitor is an inhibitor selected by
a) performing a structure based drug design using a three-dimensional structure determined for a crystal of the Adenosine Triphosphate Binding Cassette to identify a candidate inhibitor;
b) contacting the candidate inhibitor with the Adenosine Triphosphate Binding Cassette Transporter; and
c) detecting inhibition of at least one activity of the Transporter.

100. A composition in accordance with claim 97, wherein the Adenosine Triphosphate Binding Cassette Transporter is a prokaryotic Adenosine Triphosphate Binding Cassette Transporter.

101. A composition in accordance with claim 100, wherein the prokaryotic Adenosine Triphosphate Binding Cassette Transporter is a bacterial Adenosine Triphosphate Binding Cassette Transporter.

102. A composition in accordance with claim 101, wherein the bacterial Adenosine Triphosphate Binding Cassette Transporter is a gram negative bacterial Adenosine Triphosphate Binding Cassette Transporter.

103. A composition in accordance with claim 102, wherein the gram negative bacterial Adenosine Triphosphate Binding Cassette Transporter is a Salmonella Adenosine Triphosphate Binding Cassette Transporter.

104. A composition in accordance with claim 103, wherein the Salmonella Adenosine Triphosphate Binding Cassette Transporter is a Salmonella typhimurium msbA Adenosine Triphosphate Binding Cassette Transporter.

105. A composition in accordance with claim 97, wherein the inhibitor is an antibody directed against the Adenosine Triphosphate Binding Cassette Transporter.

106. A composition in accordance with claim 97, wherein the inhibitor is an antibody directed against the Salmonella typhimurium msbA Adenosine Triphosphate Binding Cassette Transporter.

107. A composition in accordance with claim 97, wherein the antibody is a monoclonal antibody.

108. A composition in accordance with claim 105, wherein the antibody is directed against at least one extracellular loop domain of the Adenosine Triphosphate Binding Cassette Transporter.

109. A composition in accordance with claim 108, wherein the at least one extracellular loop domain comprises a peptide sequence selected from the group consisting of kpllddgfgktdrsvllwmp (SEQ ID NO: 1), mfyyswqls (SEQ ID NO: 2) and asfpsvmds (SEQ ID NO: 3).

110. A composition in accordance with claim 108, wherein the antibody is directed against at least one domain of the Adenosine Triphosphate Binding Cassette Transporter which contacts a substrate.

111. A composition in accordance with claim 110, wherein the at least one domain of the Adenosine Triphosphate Binding Cassette Transporter which contacts a substrate comprises a peptide sequence selected from the group consisting of naasdtfm (SEQ ID NO: 4), gktdrsvllwmplvviglmilr (SEQ ID NO: 5) and dsltagtitvvfssm (SEQ ID NO: 6).

112. A composition in accordance with claim 105, wherein the antibody is directed against at least one intercellular domain of the Adenosine Triphosphate Binding Cassette Transporter.

113. A composition in accordance with claim 112, wherein the at least one intercellular domain comprises a peptide sequence vsgkvvmtmrrrtfghmmgmpvaffdkqstgtllsritydseqvassssga (SEQ ID NO: 7).

114. A composition in accordance with claim 105, wherein the antibody is directed against at least one drug-binding domain of the Adenosine Triphosphate Binding Cassette Transporter.

115. A composition in accordance with claim 105, wherein the antibody is directed against a peptide comprising an amino acid sequence selected from the group consisting of

| | |
|---|---|
| wqtfrrlwptiap; | (SEQ ID NO: 8) |
| glivagialiln; | (SEQ ID NO: 9) |
| litvvregasi; | (SEQ ID NO: 10) |
| iairvvsk; | (SEQ ID NO: 11) |
| sdpiiq | (SEQ ID NO: 12) |
| and | |
| mialmrplksltn. | (SEQ ID NO: 13) |

116. A composition in accordance with claim 105, wherein the antibody is directed against at least one extracellular domain of the Adenosine Triphosphate Binding Cassette Transporter.

117. A composition in accordance with claim 116, wherein the at least one extracellular domain of the Adenosine Triphosphate Binding Cassette Transporter comprises a peptide sequence selected from the group consisting of ddgfgktdrsvl (SEQ ID NO: 14), wql (SEQ ID NO: 15), and psvmdslt (SEQ ID NO: 16).

118. A composition in accordance with claim 99, wherein the inhibition of at least one activity of the Transporter comprises an inhibition of activity selected from the group consisting of ATP hydrolysis, transmembrane transport of a cationic hydrophobic compound, binding of the cationic hydrophobic compound to the Transporter and release of the cationic hydrophobic compound from the Transporter.

119. A composition in accordance with claim 118, wherein the cationic hydrophobic compound is a lipopolysaccharide.

120. A composition in accordance with claim 119, wherein the lipopolysaccharide is a rough type lipopolysaccharide selected from the group consisting of an Ra rough type lipopolysaccharide, an Rb rough type lipopolysaccharide, an Rc rough type lipopolysaccharide, an Rd rough type lipopolysaccharide and an Re rough type lipopolysaccharide.

121. A composition in accordance with claim 101, wherein the bacterial Adenosine Triphosphate Binding Cassette Transporter is a gram positive bacterial Adenosine Triphosphate Binding Cassette Transporter.

122. A composition for treating a microbial infection, the composition comprising an anti-microbial agent covalently attached to an antibody directed against an Adenosine Triphosphate Binding Cassette Transporter.

123. A composition in accordance with claim 122, wherein the anti-microbial agent is an anti-microbial peptide.

124. A composition in accordance with claim 123, wherein the anti-microbial peptide is selected from the group consisting of an insect anti-microbial peptide and a vertebrate anti-microbial peptide.

125. A composition in accordance with claim 124, wherein the insect anti-microbial peptide is a cecropin.

126. A composition in accordance with claim 125, wherein the cecropin is a cecropin-melittin hybrid peptide.

127. A composition in accordance with claim 124, wherein the vertebrate anti-microbial peptide is a mammalian anti-microbial peptide.

128. A composition in accordance with claim 123, wherein the anti-microbial peptide is selected from the group consisting of a myeloid antimicrobial peptide, an alpha-defensin, a beta-defensin, a protegrin, a porcine cecropin P1, a Bac5, a Bac7, a PR-39 and a prophenin.

129. A composition in accordance with claim 122, wherein the antibody directed against an Adenosine Triphosphate Binding Cassette Transporter is an antibody directed against a prokaryotic Adenosine Triphosphate Binding Cassette Transporter.

130. A composition in accordance with claim 129, wherein the prokaryotic Adenosine Triphosphate Binding Cassette Transporter is a bacterial Adenosine Triphosphate Binding Cassette Transporter.

131. A composition in accordance with claim 130, wherein the bacterial Adenosine Triphosphate Binding Cassette Transporter is a gram negative bacterial Adenosine Triphosphate Binding Cassette Transporter.

132. A composition in accordance with claim 131, wherein the gram negative bacterial Adenosine Triphosphate Binding Cassette Transporter is a Salmonella Adenosine Triphosphate Binding Cassette Transporter.

133. A composition in accordance with claim 132, wherein the Salmonella Adenosine Triphosphate Binding Cassette Transporter is a Salmonella typhimurium msbA Adenosine Triphosphate Binding Cassette Transporter.

134. A composition in accordance with claim 122, wherein the antibody is a monoclonal antibody.

135. A composition for treating a cancer, the composition comprising:
an inhibitor of an Adenosine Triphosphate Binding Cassette Transporter; and
an anticancer chemotherapeutic.

136. A composition in accordance with claim 135, wherein the inhibitor is an inhibitor selected by performing a structure based drug design using a three-dimensional structure determined for a crystal of the Adenosine Triphosphate Binding Cassette.

137. A composition in accordance with claim 135, wherein the inhibitor is an inhibitor selected by
a) performing a structure based drug design using a three-dimensional structure determined for a crystal of the Adenosine Triphosphate Binding Cassette to identify a candidate inhibitor;
b) contacting the candidate inhibitor with the Adenosine Triphosphate Binding Cassette Transporter; and
c) detecting inhibition of at least one activity of the Transporter.

138. A composition in accordance with claim 135, wherein the Adenosine Triphosphate Binding Cassette Transporter is a eukaryotic Adenosine Triphosphate Binding Cassette Transporter.

139. A composition in accordance with claim 138, wherein the eukaryotic Adenosine Triphosphate Binding Cassette Transporter is a mammalian Adenosine Triphosphate Binding Cassette Transporter.

140. A composition in accordance with claim 139, wherein the mammalian Adenosine Triphosphate Binding Cassette Transporter is a human Adenosine Triphosphate Binding Cassette Transporter.

141. A composition in accordance with claim 140, wherein the human Adenosine Triphosphate Binding Cassette Transporter is a P-glycoprotein Adenosine Triphosphate Binding Cassette Transporter.

142. A composition in accordance with claim 141, wherein the P-glycoprotein Adenosine Triphosphate Binding Cassette Transporter is an MDR1 Adenosine Triphosphate Binding Cassette Transporter.

143. A composition in accordance with claim 135, wherein the inhibitor is an antibody directed against the Adenosine Triphosphate Binding Cassette Transporter.

144. A composition in accordance with claim 143, wherein the inhibitor is an antibody directed against the MDR1 Adenosine Triphosphate Binding Cassette Transporter.

145. A composition in accordance with claim 143, wherein the antibody is a monoclonal antibody.

146. A composition in accordance with claim 145, wherein the antibody is directed against at least one extracellular loop domain comprised by the Adenosine Triphosphate Binding Cassette Transporter.

147. A composition in accordance with claim 143, wherein the antibody is directed against at least one domain of the Adenosine Triphosphate Binding Cassette Transporter which contacts a substrate.

148. A composition in accordance with claim 143, wherein the antibody is directed against at least one intercellular domain of the Adenosine Triphosphate Binding Cassette Transporter.

149. A composition in accordance with claim 143, wherein the antibody is directed against at least one drug-binding domain of the Adenosine Triphosphate Binding Cassette Transporter.

150. A composition in accordance with claim 143, wherein the antibody is directed against at least one extracellular domain of the Adenosine Triphosphate Binding Cassette Transporter.

151. A composition in accordance with claim 137, wherein the inhibition of at least one activity of the Transporter comprises an inhibition of an activity selected from the group consisting of ATP hydrolysis, transmembrane transport of a cationic hydrophobic compound, binding of the cationic hydrophobic compound to the Transporter and release of the cationic hydrophobic compound from the Transporter.

152. A composition in accordance with claim 151, wherein the cationic hydrophobic compound is the anticancer chemotherapeutic.

153. A composition in accordance with claim 152, wherein the anticancer chemotherapeutic is selected from the group consisting of colchicine, doxorubicin, adriamycin, vinblastine, digoxin, saquinivir and paclitaxel.

154. A composition in accordance with claim 135, wherein the inhibitor of an Adenosine Triphosphate Binding Cassette Transporter and the anticancer chemotherapeutic are covalently attached.

155. A composition for treating a microbial infection, the composition comprising:
an inhibitor of an Adenosine Triphosphate Binding Cassette Transporter covalently attached to an anti-microbial agent.

156. A composition in accordance with claim 155, wherein the inhibitor is an inhibitor selected by performing a structure based drug design using a three-dimensional structure determined for a crystal of the Adenosine Triphosphate Binding Cassette.

157. A composition in accordance with claim 155, wherein the inhibitor is an inhibitor selected by
a) performing a structure based drug design using a three-dimensional structure determined for a crystal of the Adenosine Triphosphate Binding Cassette to identify a candidate inhibitor;
b) contacting the candidate inhibitor with the Adenosine Triphosphate Binding Cassette Transporter; and
c) detecting inhibition of at least one activity of the Transporter.

158. A composition in accordance with claim 155, wherein the Adenosine Triphosphate Binding Cassette Transporter is a bacterial Adenosine Triphosphate Binding Cassette Transporter.

159. A composition in accordance with claim 158, wherein the bacterial Adenosine Triphosphate Binding Cassette Transporter is a gram negative bacterial Adenosine Triphosphate Binding Cassette Transporter.

160. A composition in accordance with claim 159, wherein the bacterial Adenosine Triphosphate Binding Cassette Transporter is a Salmonella typhimurium msbA Adenosine Triphosphate Binding Cassette Transporter.

161. A composition in accordance with claim 157, wherein the inhibition of at least one activity of the Transporter comprises an inhibition of activity selected from the group consisting of ATP hydrolysis, transmembrane transport of a cationic hydrophobic compound, binding of the cationic hydrophobic compound to the Transporter and release of the cationic hydrophobic compound from the Transporter.

162. A composition in accordance with claim 161, wherein the cationic hydrophobic compound is a lipopolysaccharide.

163. A composition in accordance with claim 162, wherein the lipopolysaccharide is a rough type lipopolysaccharide selected from the group consisting of an Ra rough type lipopolysaccharide, an Rb rough type lipopolysaccharide, an Rc rough type lipopolysaccharide, an Rd rough type lipopolysaccharide and an Re rough type lipopolysaccharide.

164. A composition in accordance with claim 158, wherein the bacterial Adenosine Triphosphate Binding Cassette Transporter is a gram positive bacterial Adenosine Triphosphate Binding Cassette Transporter.

165. A composition in accordance with claim 155, wherein the anti-microbial agent is an anti-microbial peptide.

166. A composition in accordance with claim 165, wherein the anti-microbial peptide is selected from the group consisting of an insect anti-microbial peptide and a vertebrate anti-microbial peptide.

167. A composition in accordance with claim 166, wherein the insect anti-microbial peptide is a cecropin.

168. A composition in accordance with claim 167, wherein the cecropin is a cecropin-melittin hybrid peptide.

169. A composition in accordance with claim 166, wherein the vertebrate anti-microbial peptide is a mammalian anti-microbial peptide.

170. A composition in accordance with claim 169, wherein the anti-microbial peptide is selected from the group consisting of a myeloid antimicrobial peptide, an alpha-defensin, a beta-defensin, a protegrin, a porcine cecropin P1, a Bac5, a Bac7, a PR-39 and a prophenin.

171. A therapeutic composition comprising one or more therapeutic compounds, the therapeutic compound being capable of reducing MDR that involves an Adenosine Triphosphate Binding Cassette Transporter.

172. A therapeutic composition according to claim 171, wherein the therapeutic compound is designed using structure based drug design.

173. A three dimensional computer image of the three dimensional structure of a Transporter-Ligand Complex, wherein the structure substantially conforms with the three dimensional coordinates listed in Table 1 or Table 2.

174. A computer-readable medium encoded with a set of three dimensional coordinates represented in Table 1 or Table 2, wherein, using a graphical display software program, the three dimensional coordinates create an electronic file that can be visualized on a computer capable of representing said electronic file as a three dimensional image.

175. A computer-readable medium encoded with a set of three dimensional coordinates of a three dimensional structure which substantially conforms to the three dimensional coordinates represented in Table 1 or Table 2, wherein, using a graphical display software program, the set of three dimensional coordinates create an electronic file that can be visualized on a computer capable of representing said electronic file as a three dimensional image.


SEQUENCE LISTING (33 sequences)

SEQ ID NO: 1 - protein, 20 aa, Salmonella typhimurium
KPLLDDGFGKTDRSVLLWMP

SEQ ID NO: 2 - protein, 9 aa, Salmonella typhimurium
MFYYSWQLS

SEQ ID NO: 3 - protein, 9 aa, Salmonella typhimurium
ASFPSVMDS

SEQ ID NO: 4 - protein, 8 aa, Salmonella typhimurium
NAASDTFM

SEQ ID NO: 5 - protein, 22 aa, Salmonella typhimurium
GKTDRSVLLWMPLVVIGLMILR

SEQ ID NO: 6 - protein, 15 aa, Salmonella typhimurium
DSLTAGTITVVFSSM

SEQ ID NO: 7 - protein, 51 aa, Salmonella typhimurium
VSGKVVMTMRRRLFGHMMGMPVAFFDKQSTGTLLSRITYDSEQVASSSSGA

SEQ ID NO: 8 - protein, 13 aa, Salmonella typhimurium
WQTFRRLWPTIAP

SEQ ID NO: 9 - protein, 12 aa, Salmonella typhimurium
GLIVAGIALILN

SEQ ID NO: 10 - protein, 11 aa, Salmonella typhimurium
LITVVREGASI

SEQ ID NO: 11 - protein, 8 aa, Salmonella typhimurium
IAIRVVSK

SEQ ID NO: 12 - protein, 6 aa, Salmonella typhimurium
SDPIIQ

SEQ ID NO: 13 - protein, 13 aa, Salmonella typhimurium
MIALMRPLKSLTN

SEQ ID NO: 14 - protein, 12 aa, Salmonella typhimurium
DDGFGKTDRSVL

SEQ ID NO: 15 - protein, 3 aa, Salmonella typhimurium
WQL

SEQ ID NO: 16 - protein, 8 aa, Salmonella typhimurium
PSVMDSLT

SEQ ID NO: 17 - protein, 582 aa, Salmonella typhimurium
MHNDKDLSTWQTFRRLWPTIAPFKAGLIVAGIALILNAASDTFMLSLLKPLLDDGFGKTDRSVLLWMPLVVIGLMILRGITSYISSYCISWVSGKVVMTMRRRLFGHMMGMPVAFFDKQSTGTLLSRITYDSEQVASSSSGALITVVREGASIIGLFIMMFYYSWQLSIILVVLAPIVSIAIRVVSKRFRSISKNMQNTMGQVTTSAEQMLKGHKEVLIFGGQEVETKRFDKVSNKMRLQGMKMVSASSISDPIIQLIASLALAFVLYAASFPSVMDSLTAGTITVVFSSMIALMRPLKSLTNVNAQFQRGMAACQTLFAILDSEQEKDEGKRVIDRATGDLEFRNVTFTYPGREVPALRNINLKIPAGKTVALVGRSGSGKSTIASLITRFYDIDEGHILMDGHDLREYTLASLRNQVALVSQNVHLFNDTVANNIAYARTEEYSREQIEEAARMAYAMDFINKMDNGLDTIIGENGVLLSGGQRQRIAIARALLRDSPILILDEATSALDTESERAIQAALDELQKNRTSLVIAHRLSTIEQADEIVVVEDGIIVERGTHSELLAQHGVYAQLHKMQFGQ

SEQ ID NO: 18 - protein, 582 aa, Escherichia coli
MHNDKDLSTWQTFRRLWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 19 - protein, 582 aa, Vibrio cholerae
MSLHSDESNWQTFKRLWTYIRLYKAGLVVSTIALVINAAADTYMISLLKPLLDEGFGNAESNFLRILPFMILGLMFVRGLSGFASSYCLSWVSGNVVMQMRRRLFNHFMHMPVRFFDQESTGGLLSRITYDSEQVAGATSRALVSIVREGASIIGLLTLMFWNSWQLSLVLIVVAPVVAFAISFVSKRFRKISRNMQTAMGHVTSSAEQMLKGHKVVLSYGGQEVERKRFDKVSNSMRQQTMKLVSAQSIADPVIQMIASLALFAVLFLASVDSIRAELTPGTFTVVFSAMFGLMRPLKALTSVTSEFQRGMAACQTLFGLMDLETERDNGKYEAERVNGEVDVKDVTFTYQGKEKPALSHVSFSIPQGKTVALVGRSGSGKSTIANLFTRFYDVDSGSICLDGHDVRDYKLTNLRRHFALVSQNVHLFNDTIANNIAYAAEGEYTREQIEQAARQAHAMEFIENMPQGLDTVIGENGTSLSGGQRQRVAIARALLRDAPVLILDEATSALDTESERAIQAALDELQKNKTVLVIAHRLSTIEQADEILVVDEGEIIERGRHADLLAQDGAYAQLHRIQFGE

SEQ ID NO: 20 - protein, 582 aa, Pasteurella multocida
MQDKDLSTVQTFKRLWPIISPFKLGLVVSGIALVINALADAGLISLLKPLLDEGFGKADVSFLRTMSYVVVLVIFLRGISNFISSYCLSWVSGKVVMIMRRRIFKHLMFMPVPFFDQNSSGRLLSRITYDSELVANSSSGALITIVREGAYIISLLAVMLYTSWQLSIVLFLIGPIIAVLIRFVSKRFRELSKNMQNSMGELTSTAEQMLKGHKVVLSFGGQIVEEERFNHVSNDMRRKGMKMAVADAISNPVVQIIASFALAAVLYLATVPTIMDQNLTAGSFTVVFSSMLAMMRPLKSLTNVNAQFQKGMAACQTLFALLDLETEKDLGTHKGENVQGYLSFKNVTFTYQSRDEPALRNLSFDVEKGKTVALVGRSGSGKSTIANLVTRFYDVDQGEITLDGINIQDYRLSSLRKNCAVVSQQVHLFNDTIANNIAYAAKDKYSREEIIKAAKDAYAMEFIEKLEHGLDTVIGENGVNLSGGQRQRLAIARALLRNSPVLILDEATSALDTESERSIQLALEKLQKERTVIVIAHRLSTIENADEILVIEHGEIKERGSHSELLALNGAYKQLHHIQVNH

SEQ ID NO: 21 - protein, 603 aa, Pseudomonas aeruginosa
MSDSPQNPGPSSLKIYFRLLGYVKPYIGMFLLSIVGFLIFASTQPMLAGILKYFVDGLSNPDAALFPNVQWPWLRDLHLVYAVPLLIILIAAWQGLGSFLGNFFLAKVSLGLVHDLRVALFNKLLVLPNRYFDTHSSGHLISRITFNVTMVTGAATDAIKVVIREGLTVVFLFLYLLWMNWKLTLVMLAILPVIAVMVTTASRKFRKQSKKIQVAMGDVTHVASETIQGYRVVRSFGGEAYEEKRFLDASQSNTDKQLRMTKTGAVYTPMLQLVIYVAMAILMFLVLWLRGDASAGDLVAYITAAGLLPKPIRQLSEVSSTVQRGVAGAESIFEQLDEAAEEDQGTVEKERVSGRLEVRNLSFRYPGTDKQVLDDISFIAEPGQMIALVGRSGSGKSTLANLVPRFYQHNDGKILLDGVEVEDYRLRNLRRHIALVTQQVTLFNDSVANNIAYGDLAGAPREEIERAAKAANAKEFIDNLPQGFDTEVGENGVLLSGGQRQRLAIARALLKDAPLLILDEATSALDTESERHIQAALDEVMKGRTTLVIAHRLSTIEKADLILVMDQGQIVERGSHAELLAQNGHYARLHAMGLDEQAPAPVG

SEQ ID NO: 22 - protein, 1280 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGSGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQ

SEQ ID NO: 23 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 24 - protein, 326 aa, Escherichia coli
MLTLARQQQRQNIRWLLCLSVLMLLALLLSLCAGEQWISPGDWFTPRGELFVWQIRLPRTLAVLLVGAALAISGAVMQALFENPLAEPGLLGVSNGAGVGLIAAVLLGQGQLPNWALGLCAIAGALIITLILLRFARRHLSTSRLLLAGVALGIICSALMTWAIYFSTSVDLRQLMYWMMGGFGGVDWRQSWLMLALIPVLLWICCQSRPMNMLALGEISARQLGLPLWFWRNVLVAATGWMVGVSVALAGAIGFIGLVIPHILRLCGLTDHRVLLPGCALAGASALLLADIVARLALAAAELPIGVVTATLGAPVFIWLLLKAGR

SEQ ID NO: 25 - protein, 249 aa, Escherichia coli
MSIVMQLQDVAESTRLGPLSGEVRAGEILHLVGPNGAGKSTLLARMAGMTSGKGSIQFAGQPLEAWSATKLALHRAYLSQQQTPPFAMPVWHYLTLHQHDKTRTELLNDVAGALALDDKLGRSTNQLSGGEWQRVRLAAVVLQITPQANPAGQLLLLDEPMNSLDVAQQSALDKILSALCQQGLAIVMSSHDLNHTLRHAHRAWLLKGGKMLASGRREEVLTPANLAQAYGMNFRRLDIEGHRMLISTI

SEQ ID NO: 26 - protein, 257 aa, Escherichia coli
MSENKLNVIDLHKRYGEHEVLKGVSLQANAGDVISIIGSSGSGKSTFLRCINFLEKPSEGSIVVNGQTINLVRDKDGQLKVADKNQLRLLRTRLTMVFQHFNLWSHMTVLENVMEAPIQVLGLSKQEARERAVKYLAKVGIDERAQGKYPVHLSGGQQQRVSIARALAMEPEVLLFDEPTSALDPELVGEVLRIMQQLAEEGKTMVVVTHEMGFARHVSTHVIFLHQGKIEEEGAPEQLFGNPQSPRLQQFLKGSLK

SEQ ID NO: 27 - protein, 370 aa, Escherichia coli
MASVQLQNVTKAWGEVVVSKDINLDIHEGEFVVFVGPSGCGKSTLLRMIAGLETITSGDLFIGEKRMNDTPPAERGVGMVFQSYALYPHLSVAENMSFGLKPAGAKKEVINQRVNQVAEVLQLAHLLDRKPKALSGGQRQRVAIGRTLVAEPSVFLLDEPLSNLDAALRVQMRIEISRLHKRLGRTMIYVTHDQVEAMTLADKIVVLDAGRVAQVGKPLAVPLSGRPFCRRIYRFAKDELLPVKVTATAIDQVQVELPMPNRQQVWLPVESRDVQVGANMSLGIRPEHLLPSDIADVILEGEVQVVEQLGNETQIHIQIPSIRQNLVYRQNDVVLVEEGATFAIGLPPERCHLFREDGTACRRLHKEPGV

SEQ ID NO: 28 - protein, 235 aa, Methanococcus jannaschii
MIKLKNVTKTYKMGEEIIYALKNVNLNIKEGEFVSIMGPSGSGKSTMLNIIGCLDKPTEGEVYIDNIKTNDLDDDELTKIRRDKIGFVFQQFNLIPLLTALENVELPLIFKYRGAMSGEERRKRALECLKMAELEERFANHKPNQLSGGQQQRVAIARALANNPPIILADEPTGALDSKTGEKIMQLLKKLNEEDGKTVVVVTHDINVARFGERIIYLKDGEVEREEKLRGFDDR

SEQ ID NO: 29 - protein, 583 aa, Candidatus Blochmannia floridanus
MHYCNNGNISNWKVFRRLWPIINPFKIGLIIASITLIINAVSDSLMLALLKPLLDEGFGKANRSIFMWMPLVLMGLMIMRGMSGFASTYCISWVSGKVVMHIRRLLFNHIMNMPVSFFIEQSTATLMSRITYDADQVASSSSGALITIIREGASVVGLCIMMFYYSWQLSLVLILIMPIISIIIKLVSNKFKDIGKKIQNSMSQLVNSVEQMLKGHKEVLIFGGQHLEQNRFNYLSNRMRQYTMKMVQTSSVFEPLIQFVASLALACVLYIASIPSVIEMLSAGTITVIFSSMIALMKPLKSLTNVSAQFQRGMVACQTLFTILDLKTEKNIGKFSVKRVKGHIIFDNVTFFYPETNTPSLCNINFNIESGKTIALVGRSGSGKSTIVNLLTRFYDVYQGRILLDGLNLNDYTLTSLREQVSVVTQNVYLFNDTIANNIAYARTRFYSRASIEEAAKMAYAMGFISKMKNGFDTIVGENGILLSNGQRQRIAIARALLRNCPILILDEATSSLDTESECIIYKSINILKKNRTSLIIAHRLSTIENADEILVVERGSIVERGIHVDLLNHKGVYSQLYKFQFS

SEQ ID NO: 30 - protein, 1280 aa, Canis familiaris
MDPEGGRKGSAEKNFWKMGKKSKKEKKEKKPTVSTFAMFRYSNWLDRLYMLVGTMAAIIHGAALPLMMLVFGNMTDSFANAGISRNKTFPVIINESITNNTQHFINHLEEEMTTYAYYYSGIGAGVLVAAYIQVSFWCLAAGRQILKIRKQFFHAIMRQEIGWFDVHDVGELNTRLTDDVSKINEGIGDKVGMFFQSIATFFTGFIVGFTPGWKLTLVILAISPVLGLSAAIWAKILSSFTDKELLAYAKAGAVAEEVLAAIRTVIAFGGQKKELERYNKNLEEAKRIGIKKAITANISIGAAFLLIYASYALAFWYGTSLVLSSEYTIGQVLTVFFSVLIGAFSIGQASPSIEAFANARGAAYEIFKIIDNKPSIDSYSKSGHKPDNIKGNLEFKNVHFSYPSRKEVKILKGLNLKVQSGQTVALVGNSGCGKSTTVQLMQRLYDPTDGMVCIDGQDIRTINVRHLREITGVVSQEPVLFATTIAENIRYGRENVTMDEIEKAVKEANAYDFIMKLPNKFDTLVGERGARLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTRGNEIELENATGESKSESDALEMSPKDSGSSLIKRRSTRRSIHAPQGQDRKLGTKEDLNENVPSVSFWRILKLNSTEWPYFVVGIFCAIINGGLQPAFSIIFSRIIGIFTRDEDPETKRQNSNMFSVLFLVLGIISFITFFLQGFTFGKAGEILTKRLRYMVFRSMLRQDVSWFDDPKNTTGALTTRLANDAAQVKGAIGSRLAVITQNIANLGTGIIISLIYGWQLTLLLLAIVPIIAIAGVVEMKMLSGQALKDKKELEGAGKIATEAIENFRTVVSLTREQKFEYMYAQSLQVPYRNSLRKAHIFGVSFSITQAMMYFSYAGCFRFGAYLVANEFMNFQDVLLVFSAIVFGAMAVGQVSSFAPDYAKAKVSAAHVIMIIEKSPLIDSYSPHGLKPNTLEGNVTFNEVVFNYPTRPDIPVLQGLSLEVKKGQTLALVGSSGCGKSTVVQLLERFYDPLAGSVLIDGKEIKHLNVQWLRAHLGIVSQEPILFDCSIAENIAYGDNSRVVSHEEIMQAAKEANIHHFIETLPEKYNTRVGDKGTQLSGGQKQRIAIARALVRQPHILLLDEATSALDTESEKVVQEALDKAREGRTCIVIAHRLSTIQNADLIVVFQNGKVKEHGTHQQLLAQKGIYFSMISVQAGAKR

SEQ ID NO: 31 - protein, 1276 aa, Mus musculus
MEFEENLKGRADKNFSKMGKKSKKEKKEKKPAVGVFGMFRYADWLDKLCMILGTLAAIIHGTLLPLLMLVFGNMTDSFTKAEASILPSITNQSGPNSTLIISNSSLEEEMAIYAYYYTGIGAGVLIVAYIQVSLWCLAAGRQIHKIRQKFFHAIMNQEIGWFDVHDVGELNTRLTDDVSKINDGIGDKIGMFFQSITTFLAGFIIGFISGWKLTLVILAVSPLIGLSSALWAKVLTSFTNKELQAYAKAGAVAEEVLAAIRTVIAFGGQQKELERYNKNLEEAKNVGIKKAITASISIGIAYLLVYASYALAFWYGTSLVLSNEYSIGEVLTVFFSILLGTFSIGHLAPNIEAFANARGAAFEIFKIIDNEPSIDSFSTKGYKPDSIMGNLEFKNVHFNYPSRSEVQILKGLNLKVKSGQTVALVGNSGCGKSTTVQLMQRLYDPLEGVVSIDGQDIRTINVRYLREIIGVVSQEPVLFATTIAENIRYGREDVTMDEIEKAVKEANAYDFIMKLPHQFDTLVGERGAQLSGGQKQRIAIARALVRNPKILLLDEATSALDTESEAVVQAALDKAREGRTTIVIAHRLSTVRNADVIAGFDGGVIVEQGNHDELMREKGIYFKLVMTQTRGNEIEPGNNAYGSQSDTDASELTSEESKSPLIRRSIYRSVHRKQDQERRLSMKEAVDEDVPLVSFWRILNLNLSEWPYLLVGVLCAVINGCIQPVFAIVFSRIVGVFSRDDDHETKRQNCNLFSLFFLVMGLISFVTYFFQGFTFGKAGEILTKRVRYMVFKSMLRQDISWFDDHKNSTGSLTTRLASDASSVKGAMGARLAVVTQNVANLGTGVILSLVYGWQLTLLLVVIIPLIVLGGIIEMKLLSGQALKDKKQLEISGKIATEAIENFRTIVSLTREQKFETMYAQSLQVPYRNAMKKAHVFGITFSFTQAMMYFSYAACFRFGAYLVAQQLMTFENVMLVFSAVVFGAMAAGNTSSFAPDYAKAKVSASHIIRIIEKTPEIDSYSTEGLKPTLLEGNVKFNGVQFNYPTRPNIPVLQGLSLEVKKGQTLALVGSSGCGKSTVVQLLERFYDPMAGSVFLDGKEIKQLNVQWLRAHLGIVSQEPILFDCSIAENIAYGDNSRAVSHEEIVRAAKEANIHQFIDSLPDKYNTRVGDKGTQLSGGQKQRIAIARALVRQPHILLLDEATSALDTESEKVVQEALDKAREGRTCIVIAHRLSTIQNADLIVVIENGKVKEHGTHQQLLAQKGIYFSMVQAGAKRS

SEQ ID NO: 32 - protein, 5 aa, Salmonella typhimurium
LSGGQ

SEQ ID NO: 33 - protein, 4 aa, Salmonella typhimurium
  misc_feature (2)..(2): Xaa can be any naturally occurring amino acid
RXYD